(19) United States
(12) Patent Application Publication
(10) Pub. No.: US 2016/0244494 A1
(43) Pub. Date: Aug. 25, 2016

(54) STABILIZED POLYPEPTIDES AND USES THEREOF

(71) Applicant: PRESIDENT AND FELLOWS OF HARVARD COLLEGE, Cambridge, MA (US)

(72) Inventors: Gregory L. Verdine, Boston, MA (US); Yvonne Alice Nagel, Somerville, MA (US)

(73) Assignee: President and Fellows of Harvard College, Cambridge, MA (US)

(21) Appl. No.: 15/026,473

(22) PCT Filed: Oct. 1, 2014
(86) PCT No.: PCT/US14/58680

§ 371 Date: Mar. 31, 2016

Related U.S. Application Data
(60) Provisional application No. 61/885,384, filed on Oct. 1, 2013.
(60) Provisional application No. 61/934,618, filed on Jan. 31, 2014.

(51) Int. Cl. C07K 14/47; C07C 229/30

(52) U.S. Cl. CPC C07K 14/4705; C07C 229/30; A61K 38/00

(57) ABSTRACT

The present invention provides inventive stabilized STAT polypeptides, pharmaceutical compositions thereof and methods of making and using inventive stabilized STAT polypeptides.

### RELATED APPLICATIONS

This application claims priority under 35 U.S.C. §119(e) to U.S. Provisional Patent Applications, U.S. Ser. No. 61/885,384, filed Oct. 1, 2013, and U.S. Ser. No. 61/934,618, filed Jan. 31, 2014, each of which is incorporated herein by reference.

### FIELD OF THE INVENTION

This invention relates to stabilized polypeptides and methods of treating a disease, disorder, or condition such as cancer.

### SUMMARY OF THE INVENTION

The invention provides polypeptides with more than one stabilized structural motif. In certain embodiments, the invention provides polypeptides comprising a stabilized alpha helix and an additional stabilized non-alpha helix motif (e.g., beta sheet or beta hairpin).

In certain embodiments, the invention provides polypeptides comprising a stabilized alpha helix and a stabilized beta-hairpin (stabilized α,β-motif).

In another aspect, the invention provides polypeptides comprising a stabilized alpha helix. In some embodiments, the invention provides a STAT peptide or a derivative thereof comprising a stabilized alpha.

The provided polypeptides may have good cell-penetrating capability. In certain embodiments, the provided polypeptides are capable of binding a target and/or disrupting native or aberrant protein/protein interactions. In certain embodiments, the provided polypeptides are capable of disrupting STAT protein homodimerization.

The present invention provides pharmaceutical compositions comprising a polypeptide as described herein, and optionally a pharmaceutically acceptable carrier.

In one aspect, the present invention provides a method of treating a disorder in a subject in need thereof, comprising administering an effective amount of a provided polypeptide, or pharmaceutical composition thereof, to the subject.

### DEFINITION

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March 's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, N Y, 1962); and Wilen, S. H. Tables of Resolving Agents and Optical Resolutions p. 268 (E. L. Eliel, Ed., Univ. of Notre Dame Press, Notre Dame, Ind. 1972). The invention additionally encompasses compounds as individual isomers substantially free of other isomers, and alternatively, as mixtures of various isomers.

When a range of values is listed, it is intended to encompass each value and sub-range within the range. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆alkyl.

The term “aliphatic,” as used herein, refers to alkyl, alkenyl, alkynyl, and carbocyclic groups. Likewise, the term “heteroaliphatic” as used herein, refers to heteroalkyl, heteroalkenyl, heteroalkynyl, and heterocyclic groups.

As used herein, “alkyl” refers to a radical of a straight-chain or branched saturated hydrocarbon group having from 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Unless otherwise specified, each instance of an alkyl group is independently unsubstituted (an “unsubstituted alkyl”) or substituted (a “substituted alkyl”) with one or more substituents. In certain embodiments, the alkyl group is an unsubstituted C₁₋₁₀ alkyl (e.g., —CH₃). In certain embodiments, the alkyl group is a substituted C₁₋₁₀ alkyl.

As used herein, “haloalkyl” is a substituted alkyl group as defined herein wherein one or more of the hydrogen atoms are independently replaced by a halogen, e.g., fluoro, bromo, chloro, or iodo. “Perhaloalkyl” is a subset of haloalkyl, and refers to an alkyl group wherein all of the hydrogen atoms are independently replaced by a halogen, e.g., fluoro, bromo, chloro, or iodo. In some embodiments, the haloalkyl moiety has 1 to 8 carbon atoms (“C₁₋₈ haloalkyl”). In some embodiments, the haloalkyl moiety has 1 to 6 carbon atoms (“C₁₋₆ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). In some embodiments, all of the haloalkyl hydrogen atoms are replaced with fluoro to provide a perfluoroalkyl group. In some embodiments, all of the haloalkyl hydrogen atoms are replaced with chloro to provide a “perchloroalkyl” group. Examples of haloalkyl groups include —CF₃, —CF₂CF₃, —CF₂CF₂CF₃, —CCl₃, —CFCl₂, —CF₂Cl, and the like.

As used herein, “heteroalkyl” refers to an alkyl group as defined herein which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkyl group refers to a saturated group having from 1 to 10 carbon atoms and 1 or more heteroatoms within the parent chain (“heteroC₁₋₁₀ alkyl”). In some embodiments, a heteroalkyl group is a saturated group having 1 to 9 carbon atoms and 1 or more heteroatoms within the parent chain (“heteroC₁₋₉ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 7 carbon atoms and 1 or more heteroatoms within the parent chain (“heteroC₁₋₇ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁₋₅ alkyl”). In some embodiments, a heteroalkyl group is a saturated group having 1 to 4 carbon atoms and 1 or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₀ alkyl. In certain embodiments, the heteroalkyl group is a substituted heteroC₁₋₁₀ alkyl.

As used herein, “alkenyl” refers to a radical of a straight-chain or branched hydrocarbon group having from 2 to 10 carbon atoms and one or more carbon-carbon double bonds (e.g., 1, 2, 3, or 4 double bonds). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₂₋₁₀ alkenyl. In certain embodiments, the alkenyl group is a substituted C₂₋₁₀ alkenyl.

As used herein, “heteroalkenyl” refers to an alkenyl group as defined herein which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkenyl group refers to a group having from 2 to 10 carbon atoms, at least one double bond, and 1 or more heteroatoms within the parent chain (“heteroC₂₋₁₀ alkenyl”). In some embodiments, a heteroalkenyl group has 2 to 9 carbon atoms at least one double bond, and 1 or more heteroatoms within the parent chain (“heteroC₂₋₉ alkenyl”). In some embodiments, a heteroalkenyl group has 2 to 8 carbon atoms, at least one double bond, and 1 or more heteroatoms within the parent chain (“heteroC₂₋₈ alkenyl”). In some embodiments, a heteroalkenyl group has 2 to 7 carbon atoms, at least one double bond, and 1 or more heteroatoms within the parent chain (“heteroC₂₋₇ alkenyl”). In some embodiments, a heteroalkenyl group has 2 to 6 carbon atoms, at least one double bond, and 1 or more heteroatoms within the parent chain (“heteroC₂₋₆ alkenyl”). In some embodiments, a heteroalkenyl group has 2 to 5 carbon atoms, at least one double bond, and 1 or 2 heteroatoms within the parent chain (“heteroC₂₋₅alkenyl”). In some embodiments, a heteroalkenyl group has 2 to 4 carbon atoms, at least one double bond, and 1 or 2 heteroatoms within the parent chain (“heteroC₂₋₄alkenyl”). In some embodiments, a heteroalkenyl group has 2 to 3 carbon atoms, at least one double bond, and 1 heteroatom within the parent chain (“heteroC₂₋₃ alkenyl”). In some embodiments, a heteroalkenyl group has 2 to 6 carbon atoms, at least one double bond, and 1 or 2 heteroatoms within the parent chain (“heteroC₂₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₂₋₁₀ alkenyl. In certain embodiments, the heteroalkenyl group is a substituted heteroC₂₋₁₀ alkenyl.

As used herein, “alkynyl” refers to a radical of a straight-chain or branched hydrocarbon group having from 2 to 10 carbon atoms and one or more carbon-carbon triple bonds (e.g., 1, 2, 3, or 4 triple bonds) (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₂₋₁₀ alkynyl. In certain embodiments, the alkynyl group is a substituted C₂₋₁₀ alkynyl.

As used herein, “heteroalkynyl” refers to an alkynyl group as defined herein which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkynyl group refers to a group having from 2 to 10 carbon atoms, at least one triple bond, and 1 or more heteroatoms within the parent chain (“heteroC₂₋₁₀ alkynyl”). In some embodiments, a heteroalkynyl group has 2 to 9 carbon atoms, at least one triple bond, and 1 or more heteroatoms within the parent chain (“heteroC₂₋₉ alkynyl”). In some embodiments, a heteroalkynyl group has 2 to 8 carbon atoms, at least one triple bond, and 1 or more heteroatoms within the parent chain (“heteroC₂₋₈ alkynyl”). In some embodiments, a heteroalkynyl group has 2 to 7 carbon atoms, at least one triple bond, and 1 or more heteroatoms within the parent chain (“heteroC₂₋₇ alkynyl”). In some embodiments, a heteroalkynyl group has 2 to 6 carbon atoms, at least one triple bond, and 1 or more heteroatoms within the parent chain (“heteroC₂₋₆ alkynyl”). In some embodiments, a heteroalkynyl group has 2 to 5 carbon atoms, at least one triple bond, and 1 or 2 heteroatoms within the parent chain (“heteroC₂₋₅ alkynyl”). In some embodiments, a heteroalkynyl group has 2 to 4 carbon atoms, at least one triple bond, and 1 or 2 heteroatoms within the parent chain (“heteroC₂₋₄alkynyl”). In some embodiments, a heteroalkynyl group has 2 to 3 carbon atoms, at least one triple bond, and 1 heteroatom within the parent chain (“heteroC₂₋₃ alkynyl”). In some embodiments, a heteroalkynyl group has 2 to 6 carbon atoms, at least one triple bond, and 1 or 2 heteroatoms within the parent chain (“heteroC₂₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₂₋₁₀ alkynyl. In certain embodiments, the heteroalkynyl group is a substituted heteroC₂₋₁₀ alkynyl.

As used herein, “carbocyclyl” or “carbocyclic” refers to a radical of a non-aromatic cyclic hydrocarbon group having from 3 to 10 ring carbon atoms (“C₃₋₁₀ carbocyclyl”) and zero heteroatoms in the non-aromatic ring system. In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃-carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₀ carbocyclyl. In certain embodiments, the carbocyclyl group is a substituted C₃₋₁₀ carbocyclyl.

In some embodiments, “carbocyclyl” is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₀ cycloalkyl. In certain embodiments, the cycloalkyl group is a substituted C₃₋₁₀ cycloalkyl.

As used herein, “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include, without limitation, azirdinyl, oxiranyl, thiorenyl. Exemplary 4-membered heterocyclyl groups containing 1 heteroatom include, without limitation, azetidinyl, oxetanyl and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, dioxanyl. Exemplary 6-membered heterocyclyl groups containing 2 heteroatoms include, without limitation, triazinanyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetra-hydrofuro[3,2-c]pyridinyl, 4,5,6,7-tetrahydrothieno[3,2-b]pyridinyl, 1,2,3,4-tetrahydro-1,6-naphthyridinyl, and the like.

As used herein, “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

“Aralkyl” is a subset of “alkyl” and refers to an alkyl group, as defined herein, substituted by an aryl group, as defined herein, wherein the point of attachment is on the alkyl moiety.

As used herein, “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include, without limitation, pyrrolyl, furanyl and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl and phenazinyl.

“Heteroaralkyl” is a subset of “alkyl” and refers to an alkyl group, as defined herein, substituted by a heteroaryl group, as defined herein, wherein the point of attachment is on the alkyl moiety.

As used herein, the term “partially unsaturated” refers to a ring moiety that includes at least one double or triple bond. The term “partially unsaturated” is intended to encompass rings having multiple sites of unsaturation, but is not intended to include aromatic groups (e.g., aryl or heteroaryl moieties) as herein defined.

As used herein, the term “saturated” refers to a ring moiety that does not contain a double or triple bond, i.e., the ring contains all single bonds.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

As understood from the above, alkyl, alkenyl, alkynyl, heteroalkyl, heteroalkenyl, heteroalkynyl, carbocyclyl, heterocyclyl, aryl, and heteroaryl groups, as defined herein, are, in certain embodiments, optionally substituted. Optionally substituted refers to a group which may be substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety.

Exemplary carbon atom substituents include, but are not limited to, halogen, —CN, —NO₂, —N₃, —SO₂H, —SO₃H, —OH, —OR^{aa}, —ON(R^{bb}), —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR)N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)₂R^{aa}, —OP(═O)₂R^{aa}, —P(═O)(R^{aa})₂, —OP(═O)(R^{aa})₂, —OP(═O)(OR^{cc})₂, —P(═O)₂N(R^{bb})₂, —OP(═O)₂N(R^{bb})₂, —P(═O)(NR^{bb})₂, —OP(═O)(NR^{bb})₂, —NR^{bb}P(═O)(OR^{cc})₂, —NR^{bb}P(═O)(NR^{bb})₂, —P(R^{cc})₂, —P(R^{cc})₃, —OP(R^{cc})₂, —OP(R^{cc})₃, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, C₁₋₁₀ heteroalkyl, C₂₋₁₀ heteroalkenyl, C₂₋₁₀heteroalkynyl, C₃₋₁₄ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};

each instance of R^{aa} is, independently, selected from C₁₋₁₀ alkyl, C₁₋₁₀ perhaloalkyl, C₂₋₁₀alkenyl, C₂₋₁₀ alkynyl, C₁₋₁₀ heteroalkyl, C₂₋₁₀ heteroalkenyl, C₂₋₁₀heteroalkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{aa}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀ alkenyl, C₂₋₁₀ alkynyl, C₁₋₁₀ heteroalkyl, C₂₋₁₀ heteroalkenyl, C₂₋₁₀heteroalkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, C₁₋₁₀ heteroalkyl, C₂₋₁₀ heteroalkenyl, C₂₋₁₀heteroalkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —C(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)₂R^{ee}, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, C₁₋₆ heteroalkyl, C₂₋₆ heteroalkenyl, C₂₋₆heteroalkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S;

each instance of R^{ee} is, independently, selected from C₁₋₆ alkyl, C₁₋₆perhaloalkyl, C₂₋₆ alkenyl, C₂₋₆ alkynyl, C₁₋₆ heteroalkyl, C₂₋₆ heteroalkenyl, C₂₋₆ heteroalkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;

each instance of R^{ff} is, independently, selected from hydrogen, C₁₋₆ alkyl, C₁₋₆ perhaloalkyl, C₂₋₆ alkenyl, C₂₋₆ alkynyl, C₁₋₆ heteroalkyl, C₂₋₆ heteroalkenyl, C₂₋₆heteroalkynyl, C₃₋₁₀ carbocyclyl, 3-10 membered heterocyclyl, C₆₋₁₀ aryl and 5-10 membered heteroaryl, or two R^{ff} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and

each instance of R^{gg} is, independently, halogen, —CN, —NO₂, —N₃, —SO₂H, —SO₃H, —OH, —OC₁₋₆ alkyl, —ON(C₁₋₆alkyl)₂, —N(C₁₋₆alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆alkyl), —C(═O)(C₁₋₆alkyl), —CO₂H, —CO₂(C₁₋₆alkyl), —OC(═O)(C₁₋₆ alkyl), —OCO₂(C₁₋₆ alkyl), —C(═O)NH₂, —C(═O)N(C₁₋₆alkyl)₂, —OC(═O)NH(C₁₋₆alkyl), —NHC(═O)(C₁₋₆alkyl), —N(C₁₋₆ alkyl)C(═O)(C₁₋₆ alkyl), —NHCO₂(C₁₋₆alkyl), —NHC(═O)N(C₁₋₆alkyl)₂, —NHC(═O)NH(C₁₋₆ alkyl), —NHC(═O)NH₂, —C(═NH)O(C₁₋₆alkyl), —OC(═NH)(C₁₋₆ alkyl), —OC(═NH)OC₁₋₆alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆alkyl, —OSO₂C₁₋₆ alkyl, —SOC₁₋₆alkyl, —Si(C₁₋₆ alkyl)₃, —OSi(C₁₋₆ alkyl)₃-C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)₂(C₁₋₆ alkyl), —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆perhaloalkyl, C₂₋₆ alkenyl, C₂₋₆ alkynyl, C₁₋₆heteroalkyl, C₂₋₆ heteroalkenyl, C₂₋₆heteroalkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

As used herein, the term “halo” or “halogen” refers to fluorine (fluoro, —F), chlorine (chloro, —Cl), bromine (bromo, —Br), or iodine (iodo, —I).

As used herein, the term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃, —OP(═O)₂R^{aa}, —OP(═O)(R^{aa})₂, —OP(═O)(OR^{cc})₂, —OP(═O)₂N(R^{bb})₂, and —OP(═O)(NR^{bb})₂, wherein R^{aa}, R^{bb}, and R^{cc} are as defined herein.

As used herein, the term “thiol” or “thio” refers to the group —SH. The term “substituted thiol” or “substituted thio,” by extension, refers to a thiol group wherein the sulfur atom directly attached to the parent molecule is substituted with a group other than hydrogen, and includes groups selected from —SR^{aa}, —S═SR^{cc}, —SC(═S)SR^{aa}, —SC(═O)SR^{aa}, —SC(═O)OR^{aa}, and —SC(═O)R^{aa}, wherein R^{aa} and R^{cc} are as defined herein.

As used herein, the term, “amino” refers to the group —NH₂. The term “substituted amino,” by extension, refers to a monosubstituted amino, a disubstituted amino, or a trisubstituted amino, as defined herein. In certain embodiments, the “substituted amino” is a monosubstituted amino or a disubstituted amino group.

As used herein, the term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(NR^{bb})₂, wherein R^{aa}, R^{bb} and R^{cc} are as defined herein, and wherein R^{bb} of the group —NH(R^{bb}) is not hydrogen.

As used herein, the term “disubstituted amino” refers to an amino group wherein the nitrogen atom directly attached to the parent molecule is substituted with two groups other than hydrogen, and includes groups selected from —N(R^{bb})₂, —NR^{bb} C(═O)R^{aa}, —NR^{bb}CO₂R^{aa}, —NR^{bb}C(═O)N(R^{bb})₂, —NR^{bb}C(═NR^{bb})N(R^{bb})₂, —NR^{bb}SO₂R^{aa}, —NR^{bb}P(═O)(OR^{cc})₂, and —NR^{bb}P(═O)(NR^{bb})₂, wherein R^{aa}, R^{bb}, and R^{cc} are as defined herein, with the proviso that the nitrogen atom directly attached to the parent molecule is not substituted with hydrogen.

As used herein, the term “trisubstituted amino” refers to an amino group wherein the nitrogen atom directly attached to the parent molecule is substituted with three groups, and includes groups selected from —N(R^{bb})₃ and —N(R^{bb})₃⁺X⁻, wherein R^{bb} and X⁻ are as defined herein.

As used herein, the term “oxo” refers to the group ═O, and the term “thiooxo” refers to the group ═S.

Nitrogen atoms can be substituted or unsubstituted as valency permits, and include primary, secondary, tertiary, and quarternary nitrogen atoms. Exemplary nitrogen atom substitutents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR)OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀ alkenyl, C₂₋₁₀ alkynyl, C₁₋₁₀ heteroalkyl, C₂₋₁₀ heteroalkenyl, C₂₋₁₀ heteroalkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, the substituent present on the nitrogen atom is an nitrogen protecting group (also referred to herein as an “amino protecting group”). Nitrogen protecting groups include, but are not limited to, —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂R^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₂₋₁₀ alkenyl, C₂₋₁₀ alkynyl, C₁₋₁₀ heteroalkyl, C₂₋₁₀ heteroalkenyl, C₂₋₁₀ heteroalkynyl, C₃₋₁₀carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, nitrogen protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide and o-(benzoyloxymethyl)benzamide.

Nitrogen protecting groups such as carbamate groups (e.g., —C(═O)OR^{aa}) include, but are not limited to, methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-0,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1, 1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Teroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Nitrogen protecting groups such as sulfonamide groups (e.g., —S(═O)₂R^{aa}) include, but are not limited to, p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, the substituent present on an oxygen atom is an oxygen protecting group (also referred to herein as an “hydroxyl protecting group”). Oxygen protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃, —P(═O)₂R^{aa}, —P(═O)(R^{aa})₂, —P(═O)(OR^{cc})₂, —P(═O)₂N(R^{bb})₂, and —P(═O)(NR^{bb})₂, wherein R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include, but are not limited to, methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkylp-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, the substituent present on an sulfur atom is a sulfur protecting group (also referred to as a “thiol protecting group”). Sulfur protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃, —P(═O)₂R^{aa}, —P(═O)(R^{aa})₂, —P(═O)(OR^{cc})₂, —P(═O)₂N(R^{bb})₂, and —P(═O)(NR^{bb})₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

These and other exemplary substituents are described in more detail in the Detailed Description, Examples, and claims. The invention is not intended to be limited in any manner by the above exemplary listing of substituents.

As used herein, the term “salt” refers to any and all salts.

The term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

As used herein, the term “tautomer” refers to particular isomers of a compound in which a hydrogen and double bond have changed position with respect to the other atoms of the molecule. For a pair of tautomers to exist there must be a mechanism for interconversion. Examples of tautomers include keto-enol forms, imine-enamine forms, amide-imino alcohol forms, amidine-aminidine forms, nitroso-oxime forms, thio ketone-enethiol forms, N-nitroso-hydroxyazo forms, nitro-aci-nitro forms, and pyridone-hydroxypyridine forms.

As used herein, use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

As used herein, the term “leaving group” is given its ordinary meaning in the art of synthetic organic chemistry and refers to an atom or a group capable of being displaced by a nucleophile. Examples of suitable leaving groups include, but are not limited to, halides (such as chloride, bromide, or iodide), alkoxycarbonyloxy, aryloxycarbonyloxy, alkanesulfonyloxy, arenesulfonyloxy, alkyl-carbonyloxy (e.g., acetoxy), arylcarbonyloxy, aryloxy, methoxy, N,O-dimethylhydroxylamino, pixyl, haloformates, —NO₂, trialkylammonium, and aryliodonium salts. In some embodiments, the leaving group is a sulfonic acid ester. In some embodiments, the sulfonic acid ester comprises the formula —OSO₂R′ wherein R′ is selected from the group consisting alkyl optionally, alkenyl optionally substituted, heteroalkyl optionally substituted, aryl optionally substituted, heteroaryl optionally substituted, arylalkyl optionally substituted, and heterarylalkyl optionally substituted. In some embodiments, R′ is substituted or unsubstituted C₁-C₆ alkyl. In some embodiments, R′ is methyl. In some embodiments, R′ is —CF₃. In some embodiments, R′ is substituted or unsubstituted aryl. In some embodiments, R′ is substituted or unsubstituted phenyl. In some embodiments R′ is:

In some cases, the leaving group is toluenesulfonate (tosylate, Ts), methanesulfonate (mesylate, Ms), p-bromobenzenesulfonyl (brosylate, Bs), or trifluoromethanesulfonate (triflate, Tf). In some cases, the leaving group is a brosylate (p-bromobenzenesulfonyl). In some cases, the leaving group is a nosylate (2-nitrobenzenesulfonyl). In some embodiments, the leaving group is a sulfonate-containing group. In some embodiments, the leaving group is a tosylate group. The leaving group may also be a phosphineoxide (e.g., formed during a Mitsunobu reaction) or an internal leaving group such as an epoxide or cyclic sulfate.

The term “amino acid” refers to a molecule containing both an amino group and a carboxyl group. Amino acids include alpha-amino acids and beta-amino acids, the structures of which are depicted below. In certain embodiments, an amino acid is an alpha amino acid.

Suitable amino acids include, without limitation, natural alpha-amino acids such as D- and L-isomers of the 20 common naturally occurring alpha-amino acids found in peptides (e.g., A, R, N, C, D, Q, E, G, H, I, L, K, M, F, P, S, T, W, Y, V, as provided in Table 1 depicted below), unnatural alpha-amino acids (as depicted in Tables 2 and 3 below), natural beta-amino acids (e.g., beta-alanine), and unnatural beta-amino acids.

Amino acids used in the construction of peptides of the present invention may be prepared by organic synthesis, or obtained by other routes, such as, for example, degradation of or isolation from a natural source. In certain embodiments of the present invention, the formula —[X_{AA}]— corresponds to the natural and/or unnatural amino acids having the following formulae:

wherein R and R′ correspond a suitable amino acid side chain, as defined below and herein, and R^{a} is as defined below and herein.

TABLE 1

| Exemplary natural alpha- | Suitable amino acid side chains | |
|---|---|---|
| amino acids | R | R′ |
| L-Alanine (A) | —CH₃ | —H |
| L-Arginine (R) | —CH₂CH₂CH₂—NHC(═NH)NH₂ | —H |
| L-Asparagine (N) | —CH₂C(═O)NH₂ | —H |
| L-Aspartic acid (D) | —CH₂CO₂H | —H |
| L-Cysteine (C) | —CH₂SH | —H |
| L-Glutamic acid (E) | —CH₂CH₂CO₂H | —H |
| L-Glutamine (Q) | —CH₂CH₂C(═O)NH₂ | —H |
| Glycine (G) | —H | —H |
| L-Histidine (H) | —CH₂-2-(1H-imidazole) | —H |
| L-Isoleucine (I) | -sec-butyl | —H |
| L-Leucine (L) | -iso-butyl | —H |
| L-Lysine (K) | —CH₂CH₂CH₂CH₂NH₂ | —H |
| L-Methionine (M) | —CH₂CH₂SCH₃ | —H |
| L-Phenylalanine (F) | —CH₂Ph | —H |
| L-Proline (P) | -2-(pyrrolidine) | —H |
| L-Serine (S) | —CH₂OH | —H |
| L-Threonine (T) | —CH₂CH(OH)(CH₃) | —H |
| L-Tryptophan (W) | —CH₂-3-(1H-indole) | —H |
| L-Tyrosine (Y) | —CH₂-(p-hydroxyphenyl) | —H |
| L-Valine (V) | -isopropyl | —H |

TABLE 2

| Exemplary unnatural | | |
|---|---|---|
| alpha-amino acids | R | R′ |
| | Suitable amino acid side chains | |
| D-Alanine | —H | —CH₃ |
| D-Arginine | —H | —CH₂CH₂CH₂—NHC(═NH)NH₂ |
| D-Asparagine | —H | —CH₂C(═O)NH₂ |
| D-Aspartic acid | —H | —CH₂CO₂H |
| D-Cysteine | —H | —CH₂SH |
| D-Glutamic acid | —H | —CH₂CH₂CO₂H |
| D-Glutamine | —H | —CH₂CH₂C(═O)NH₂ |
| D-Histidine | —H | —CH₂-2-(1H-imidazole) |
| D-Isoleucine | —H | -sec-butyl |
| D-Leucine | —H | -iso-butyl |
| D-Lysine | —H | —CH₂CH₂CH₂CH₂NH₂ |
| D-Methionine | —H | —CH₂CH₂SCH₃ |
| D-Phenylalanine | —H | —CH₂Ph |
| D-Proline | —H | -2-(pyrrolidine) |
| D-Serine | —H | —CH₂OH |
| D-Threonine | —H | —CH₂CH(OH)(CH₃) |
| D-Tryptophan | —H | —CH₂-3-(1H-indole) |
| D-Tyrosine | —H | —CH₂-(p-hydroxyphenyl) |
| D-Valine | —H | -isopropyl |
| Di-vinyl | —CH═CH₂ | —CH═CH₂ |
| | R and R′ are equal to: | |
| α-methyl-Alanine | —CH₃ | —CH₃ |
| (Aib) | | |
| α-methyl-Arginine | —CH₃ | —CH₂CH₂CH₂—NHC(═NH)NH₂ |
| α-methyl-Asparagine | —CH₃ | —CH₂C(═O)NH₂ |
| α-methyl-Aspartic | —CH₃ | —CH₂CO₂H |
| acid | | |
| α-methyl-Cysteine | —CH₃ | —CH₂SH |
| α-methyl-Glutamic | —CH₃ | —CH₂CH₂CO₂H |
| acid | | |
| α-methyl-Glutamine | —CH₃ | —CH₂CH₂C(═O)NH₂ |
| α-methyl-Histidine | —CH₃ | —CH₂-2-(1H-imidazole) |
| α-methyl-Isoleucine | —CH₃ | -sec-butyl |
| α-methyl-Leucine | —CH₃ | -iso-butyl |
| α-methyl-Lysine | —CH₃ | —CH₂CH₂CH₂CH₂NH₂ |
| α-methyl-Methionine | —CH₃ | —CH₂CH₂SCH₃ |
| α-methyl- | —CH₃ | —CH₂Ph |
| Phenylalanine | | |
| α-methyl-Proline | —CH₃ | -2-(pyrrolidine) |
| α-methyl-Serine | —CH₃ | —CH₂OH |
| α-methyl-Threonine | —CH₃ | —CH₂CH(OH)(CH₃) |
| α-methyl-Tryptophan | —CH₃ | —CH₂-3-(1H-indole) |
| α-methyl-Tyrosine | —CH₃ | —CH₂-(p-hydroxyphenyl) |
| α-methyl-Valine | —CH₃ | -isopropyl |
| Di-vinyl | —CH═CH₂ | —CH═CH₂ |
| Norleucine | —H | —CH₂CH₂CH₂CH₃ |

TABLE 3

| | Suitable amino acid side chains |
|---|---|
| Exemplary unnatural | R and R′ is equal to hydrogen or |
| alpha-amino acids | —CH₃, and: |
| Terminally unsaturated | —(CH₂)_{g}—S—(CH₂)_{g}CH═CH₂, |
| alpha-amino acids and bis | —(CH₂)_{g}—O—(CH₂)_{g}CH═CH₂, |
| alpha-amino acids(e.g., | —(CH₂)_{g}—NH—(CH₂)_{g}CH═CH₂, |
| modified cysteine, | —(CH₂)_{g}—(C═O)—S—(CH₂)_{g}CH═CH₂, |
| modified lysine, | —(CH₂)_{g}—(C═O)—O—(CH₂)_{g}CH═CH₂, |
| modified tryptophan, | —(CH₂)_{g}—(C═O)—NH—(CH₂)_{g}CH═CH₂, |
| modified serine, | —CH₂CH₂CH₂CH₂—NH—(CH₂)_{g}CH═CH₂, |
| modified threonine, | —(C₆H₅)-p-O—(CH₂)_{g}CH═CH₂, |
| modified proline, | —CH(CH₃)—O—(CH₂)_{g}CH═CH₂, |
| modified histidine, | —CH₂CH(—O—CH═CH₂)(CH₃), |
| modified alanine, | -histidine-N((CH₂)_{g}CH═CH₂), |
| and the like). | -tryptophan-N((CH₂)_{g}CH═CH₂), and |
| | —(CH₂)_{g+1}(CH═CH₂), |
| | wherein: |
| | each instance of g is, |
| | independently, 0 to 10. |

```
Exemplary unnatural alpha-amino acids
  R₅
  R₈
  S₅
  S₈
  B₅
```

There are many known unnatural amino acids any of which may be included in the peptides of the present invention. See for example, S. Hunt, The Non-Protein Amino Acids: In Chemistry and Biochemistry of the Amino Acids, edited by G. C. Barrett, Chapman and Hall, 1985. Some examples of unnatural amino acids are 4-hydroxyproline, desmosine, gamma-aminobutyric acid, beta-cyanoalanine, norvaline, 4-(E)-butenyl-4(R)-methyl-N-methyl-L-threonine, N-methyl-L-leucine, 1-amino-cyclopropanecarboxylic acid, 1-amino-2-phenyl-cyclopropanecarboxylic acid, 1-amino-cyclobutanecarboxylic acid, 4-amino-cyclopentenecarboxylic acid, 3-amino-cyclohexanecarboxylic acid, 4-piperidylacetic acid, 4-amino-1-methylpyrrole-2-carboxylic acid, 2,4-diaminobutyric acid, 2,3-diaminopropionic acid, 2,4-diaminobutyric acid, 2-aminoheptanedioic acid, 4-(aminomethyl)benzoic acid, 4-aminobenzoic acid, ortho-, meta- and para-substituted phenylalanines (e.g., substituted with —C(═O)C₆H₅; —CF₃; —CN; -halo; —NO₂; CH₃), disubstituted phenylalanines, substituted tyrosines (e.g., further substituted with —C(═O)C₆H₅; —CF₃; —CN; -halo; —NO₂; CH₃), and statine. Additionally, the amino acids suitable for use in the present invention may be derivatized to include amino acid residues that are hydroxylated, phosphorylated, sulfonated, acylated, and glycosylated, to name a few.

The term “amino acid side chain” refers to a group attached to the alpha- or beta-carbon of an amino acid. A “suitable amino acid side chain” includes, but is not limited to, any of the suitable amino acid side chains as defined above, and as provided in Tables 1 to 3.

For example, suitable amino acid side chains include methyl (as the alpha-amino acid side chain for alanine is methyl), 4-hydroxyphenylmethyl (as the alpha-amino acid side chain for tyrosine is 4-hydroxyphenylmethyl) and thiomethyl (as the alpha-amino acid side chain for cysteine is thiomethyl), etc. A “terminally unsaturated amino acid side chain” refers to an amino acid side chain bearing a terminal unsaturated moiety, such as a substituted or unsubstituted, double bond (e.g., olefinic) or a triple bond (e.g. acetylenic), that participates in crosslinking reaction with other terminal unsaturated moieties in the polypeptide chain. In certain embodiments, a “terminally unsaturated amino acid side chain” is a terminal olefinic amino acid side chain. In certain embodiments, a “terminally unsaturated amino acid side chain” is a terminal acetylenic amino acid side chain. In certain embodiments, the terminal moiety of a “terminally unsaturated amino acid side chain” is not further substituted. Terminally unsaturated amino acid side chains include, but are not limited to, side chains as depicted in Table 3.

A “peptide” or “polypeptide” comprises a polymer of amino acid residues linked together by peptide (amide) bonds. The term(s), as used herein, refers to proteins, polypeptides, and peptide of any size, structure, or function. Typically, a peptide or polypeptide will be at least three amino acids long. A peptide or polypeptide may refer to an individual protein or a collection of proteins. Inventive proteins preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in a peptide or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A peptide or polypeptide may also be a single molecule or may be a multi-molecular complex, such as a protein. A peptide or polypeptide may be just a fragment of a naturally occurring protein or peptide. A peptide or polypeptide may be naturally occurring, recombinant, or synthetic, or any combination thereof. As used herein “dipeptide” refers to two covalently linked amino acids.

“Peptide stapling” refers to cross-linking side chains of a polypeptide chain by covalently joining olefin moieties (i.e., “stapled together”) using a ring-closing metathesis (RCM) reaction. “Peptide stitching” encompasses multiple “staples” in a single polypeptide chain to provide a multiply stapled (also known as “stitched”) polypeptide (see U.S. Pat. Nos. 7,192,713 and 7,786,072, and International PCT Publications WO2008/121767 and WO2011/008260, each of which is incorporated herein by reference).

As generally used herein, the RCM reaction refers to formation of alkenyl or alkynyl cross-linkers in the polypeptide with an RCM catalyst. A suitable RCM catalyst is a tungsten (W), molybdenum (Mo), or ruthenium (Ru) catalyst. In certain embodiments, the RCM catalyst is a ruthenuim catalyst. Examples of suitable olefin metathesis catalyst include, but are not limited to, Schrock catalyst, Grubbs Catalyst 1st generation, or benzylidene-bis(tricyclohexylphosphine)dichlororuthenium, Grubbs Catalyst 2nd Generation, or benzylidene[1,3-bis(2,4,6-trimethylphenyl)-2-midazolidinylidene]dichloro-(tricyclohexylphosphine)ruthenium, and Hoveyda-Grubbs Catalyst 2nd Generation, or 1,3-bis-(2,4,6-trimethylphenyl)-2-imidazolidinylidene)dichloro(o-isopropoxyp-phenylmethylene)ruthenium. RCM catalysts employable by the above synthetic method are described in Grubbs et al., Acc. Chem. Res. 1995, 28, 446-452, U.S. Pat. No. 5,811,515; Schrock et al., Organometallics (1982) 1 1645; Gallivan et al., Tetrahedron Letters (2005) 46:2577-2580; Furstner et al., J. Am. Chem. Soc. (1999) 121:9453; and Chem. Eur. J. (2001) 7:5299; WO2008/121767 and WO2011/008260; the entire contents of each of which are incorporated herein by reference

As generally used herein, the click chemistry reaction is a chemical approach to generate substances quickly and reliably by joining small units together. See, e.g., Kolb, Finn and Sharpless, Angewandte Chemie International Edition (2001) 40: 2004-2021; Evans, Australian Journal of Chemistry (2007) 60: 384-395; all of which are incorporated by reference herein). Exemplary coupling reactions (some of which may be classified as “click chemistry”) include, but are not limited to, formation of esters, thioesters, amides (e.g., such as peptide coupling) from activated acids or acyl halides; nucleophilic displacement reactions (e.g., such as nucleophilic displacement of a halide or ring opening of strained ring systems); azide-alkyne Huisgon cycloaddition; thiol-yne addition; imine formation; and Michael additions (e.g., maleimide addition). In certain embodiments, the click chemistry reaction used in the present invention is azide alkyne Huisgen cycloaddition (Rostovtsev et al., Angewandte Chemie International Edition, 41 (14): 2596-2599). In certain embodiments, copper reagents such as reagents which provide a reactive Cu(I) species, such as CuBr, CuI or CuOTf, as well as Cu(II) salts such as Cu(CO₂CH₃)₂, CuSO₄, and CuCl₂ that can be converted in situ to an active Cu(I) reagent by the addition of a reducing agent such as ascorbic acid or sodium ascorbate, can be present in the click reaction.

An “effective amount” refers to an amount sufficient to elicit a desired biological response, i.e., treating the condition. As will be appreciated by those of ordinary skill in this art, the effective amount of a provided polypeptide may vary depending on such factors as the desired biological endpoint, the pharmacokinetics of the polypeptide, the condition being treated, the mode of administration, and the age and health of the subject. An effective amount encompasses therapeutic and prophylactic treatment. For example, in treating cancer, an effective amount of an inventive polypeptide may reduce the tumor burden or stop the growth or spread of a tumor.

As used herein, the terms “treatment,” “treat,” and “treating” refer to reversing, alleviating, delaying the onset of, or inhibiting the progress of a “pathological condition” (e.g., a disease, disorder, or condition, or one or more signs or symptoms thereof) described herein. In some embodiments, treatment may be administered after one or more signs or symptoms have developed or have been observed. In other embodiments, treatment may be administered in the absence of signs or symptoms of the disease or condition. For example, treatment may be administered to a susceptible individual prior to the onset of symptoms (e.g., in light of a history of symptoms and/or in light of genetic or other susceptibility factors). Treatment may also be continued after symptoms have resolved, for example, to delay or prevent recurrence.

As used herein “inhibition”, “inhibiting”, “inhibit” and “inhibitor”, and the like, refer to the ability of a polypeptide to reduce, slow, halt, or prevent the activity of a particular biological process involving STAT in a cell relative to vehicle.

### DETAILED DESCRIPTION OF THE FIGURES

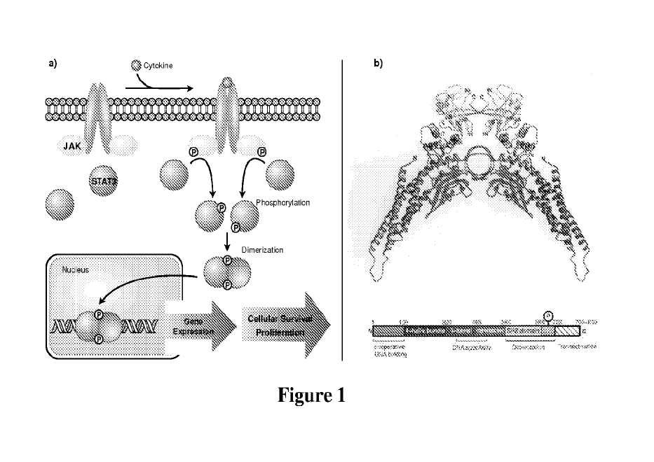

FIG. 1a shows schematic representation of the JAK/STAT pathway (layout adapted from D. Leroith, P. Nissley, The Journal of clinical investigation 2005, 115, 233-236). FIG. 1b shows the crystal structure of the STAT33 homodimer-DNA complex (view along the DNA axis) and domain structure (S. Becker, B. Groner, C. W. Muller, Nature 1998, 394, 145-151).

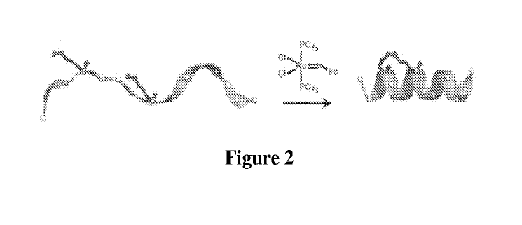

FIG. 2 shows exemplary Ruthenium-mediated ring-closing metathesis with Grubbs first generation catalyst enforces the unstructured peptide fragment into a stabilized α-helix.

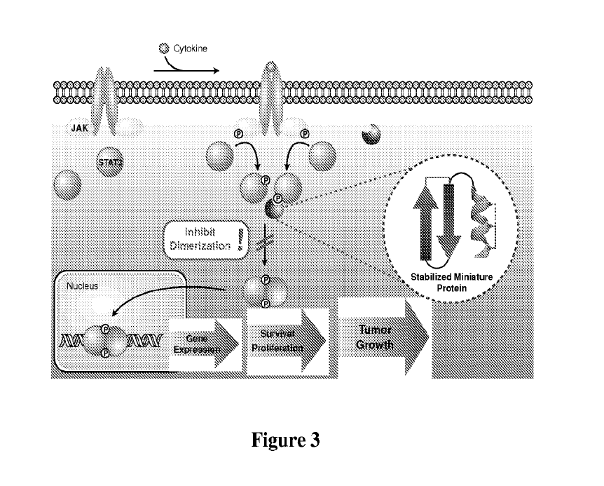

FIG. 3 shows inhibition of STAT3 dimerization within the JAK/STAT pathway by a stabilized miniature protein derived from the SH2 domain of STAT3 as described in the present invention (layout adapted from D. Leroith, P. Nissley, The Journal of clinical investigation 2005, 115, 233-236).

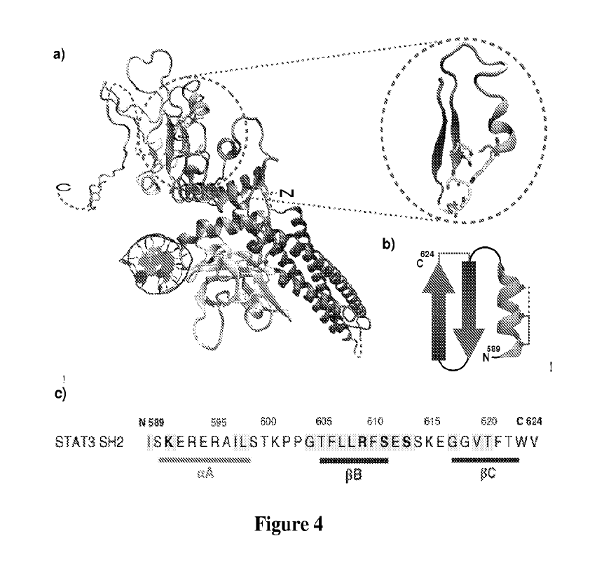

FIG. 4a shows the crystal structure of STAT3 bound to DNA (grey, only one monomer shown) with excised residues 589-624 displaying an α-helix (green) flanked by a β-hairpin (blue) (adapted from PDB file 1BG1, S. Becker, B. Groner, C. W. Muller, Nature 1998, 394, 145-151. FIG. 4b shows the schematic representation of the excised residues 589-624 with potential synthetic stabilizations (red). FIG. 4c shows the excised motif for the synthesis of a stabilized α,β-motif. Residues involved in phosphotyrosine binding are represented in bold black letters and conserved residues are shadowed.

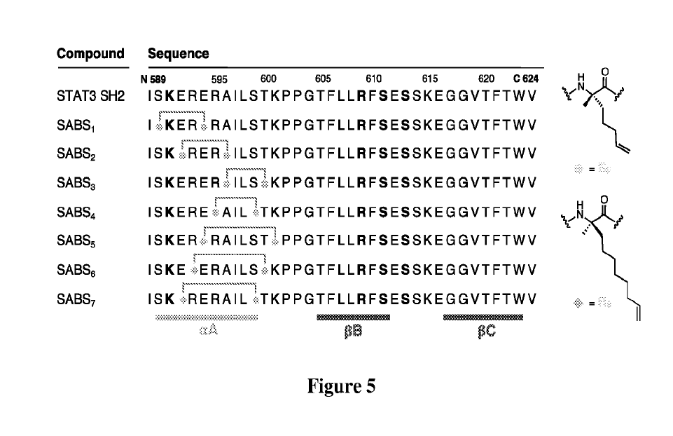

FIG. 5 shows stabilized α-helix and β-hairpin peptides of STAT3 SH2 (SABS) having i,i+4 and i,i+7 staples. Modified amino acids (green dots and diamonds) for formation of stabilizing elements in distinct positions are indicated.

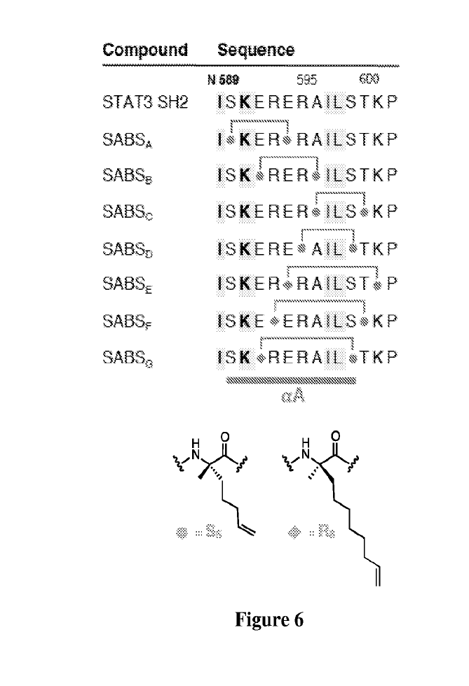

FIG. 6 shows all-hydrocarbon stapled peptides of the α-helical portion of the STAT3 SH2 motif with i,i+4 and i,i+7 staples.

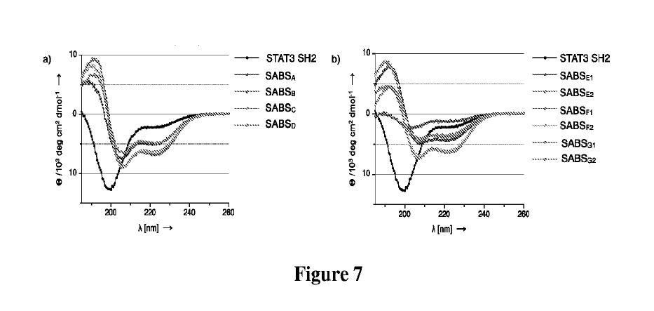

FIG. 7 shows CD spectra of all-hydrocarbon stapled peptides of the a-helical portion of the STAT3 SH2 motif with i,i+4 (left) and i,i+7 (right) staples including cis/trans isomers of the olefin (e.g., SABS_{E1} and SABS_{E2}), compared to the wildtype STAT3 SH2 peptide (black). The CD spectra were recorded in Milli Q water, pH 5.5, 100 μM, 20° C. The stapled peptides show a-helical characteristics compared to wild type STAT3 SH2.

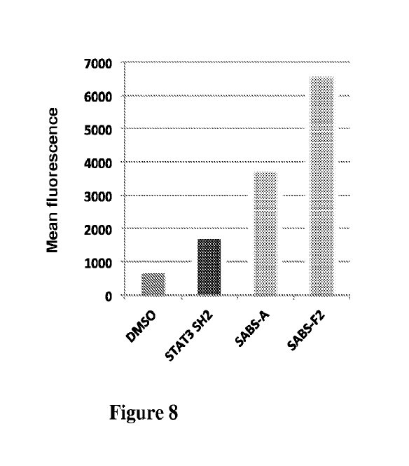

FIG. 8 shows mean cellular fluorescence of Jurkat cells incubated for 3 h at 37° C. with 5 μM fluorescently SABS-A and SABS-F2, compared to wildtype STAT3 SH2 peptide and DMSO as negative control.

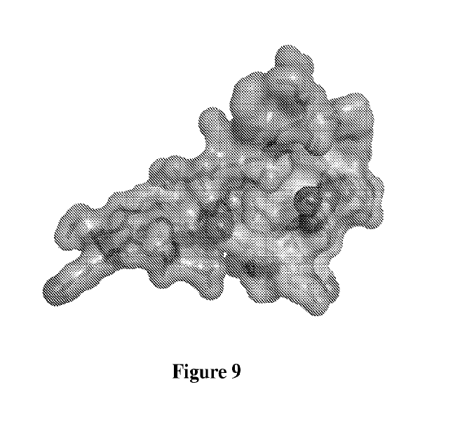

FIG. 9 shows α-Helix (green) and β-hairpin (light blue) surfaces interacting with each other in the desired α,β-motif of the STAT3 SH2 domain (generated from 1BG1). Based on polar and hydrophobic interactions of the α-helix with the β-hairpin found in the crystal structure of the STAT3 SH2 domain, a templating effect of the α-helix may support the folding of the β-hairpin segment and thus lead to an additional stabilization of the structure of the desired miniature proteins.

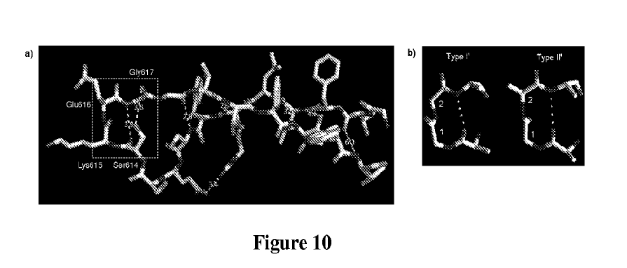

FIG. 10a shows the crystal structure of the wildtype β-hairpin segment (strands βB and βC) of the STAT3 SH2 domain in the STAT3 dimer bound to DNA (1BG1). FIG. 10b shows the two-residue β-hairpin turns, white dots indicate hydrogen bonds (J. Cooper, http://www.cryst.bbk.ac.uk/PPS2/course/section9/sss/super2.html 1996). The unmodified β-hairpin segment reveals that the two residues that induce the β-turn (Lys615-Glu616) form a type II′ turn conformation, with the carbonyl group between them pointing backwards in the given orientation (FIG. 10a). The main difference between type I′ and type II′ turns in general is the orientation of this specific carbonyl group of the amide bond between the two amino acids (residues 1 and 2 in FIG. 10b) (J. Cooper, www.cryst.bbk.ac.uk/PPS2/course/section9/sss/super2.html 1996). A type II′ β-turn in a α-hairpin conformation can be induced by exploiting the nucleation effect of a heterochiral D-Pro-L-Pro (pP) dipeptide template (Aravinda, U. S. Raghavender, R. Rai, V. V. Harini, N. Shamala, P. Balaram, Organic & Biomolecular Chemistry 2013, 11, 4220-4231; J. Späth, F. Stuart, L. Jiang, J. A. Robinson, Helvetica Chimica Acta 1998, 81, 1726-1738). Although the adjacent carbonyl group of Ser614 that is supposed to form the hydrogen bond in a pure two-residue β-turn does not show the optimal geometry and distance for hydrogen bonding to the secondary amine on the opposite side of the structure, it appears that the hydroxyl group of the Ser614 instead could act as a stabilizing element by interacting with the carbonyl group of Gly617. This arrangement displays a tendency towards a three-residue β-hairpin turn, which could in addition be stabilized by insertion of a D-Pro-L-Pro-D-Ala tripeptide motif within the sequence (R. Rai, S. Raghothama, P. Balaram, Journal of the American Chemical Society 2006, 128, 2675-2681).

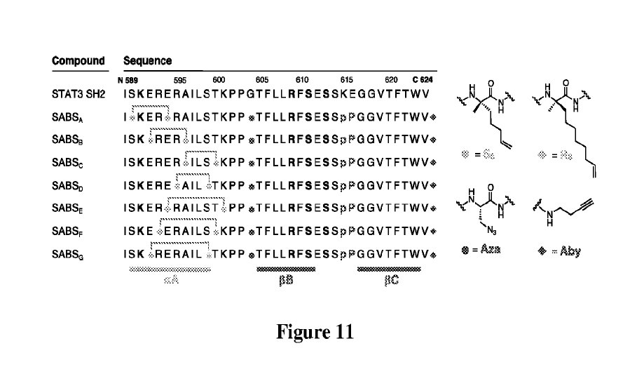

FIG. 11 shows stabilized α-helix and 1-hairpin peptides of STAT3 SH2 (SABS) with positions for i,i+4 and i,i+7 staples, as well as D-Pro-L-Pro (pP) motif for inducing a β-turn. Modified amino acids (green and blue dots and diamonds) for formation of stabilizing elements in distinct positions are indicated. A D-Pro-L-Pro motif and alternatively a D-Pro-L-Pro-D-Ala template, as well as the incorporation of the modified building block (-azidoalanine (Aza), are introduced into the sequence. Eventually 1-amino-3-butyne (Aby) is C-terminally coupled after cleavage from solid support, allowing for a Cu(I)-catalyzed 1,3-dipolar azide alkyne cycloaddition (“click reaction”) to form a cross-link for β-hairpin stabilization.

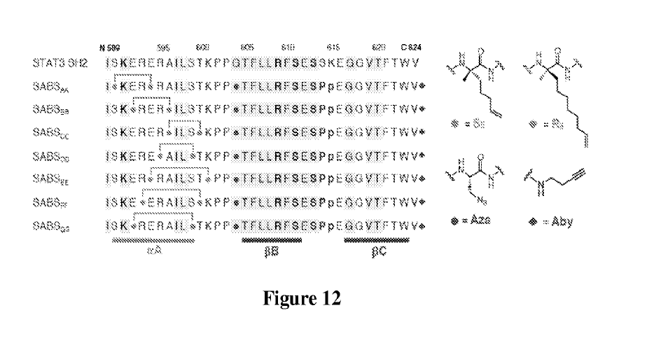

FIG. 12 shows stabilized α-helix and β-hairpin peptides of STAT3 SH2 (SABS) with positions for i,i+4 and i,i+7 staples, as well as D-Pro-L-Pro (pP) motif for inducing a β-turn. Modified amino acids (green and blue dots and diamonds) for formation of stabilizing elements in distinct positions are indicated. A D-Pro-L-Pro motif and alternatively a D-Pro-L-Pro-D-Ala template, as well as the incorporation of the modified building block β-azidoalanine (Aza), are introduced into the sequence. Eventually 1-amino-3-butyne (Aby) is C-terminally coupled after cleavage from solid support, allowing for a Cu(I)-catalyzed 1,3-dipolar azide alkyne cycloaddition (“click reaction”) to form a cross-link for β-hairpin stabilization.

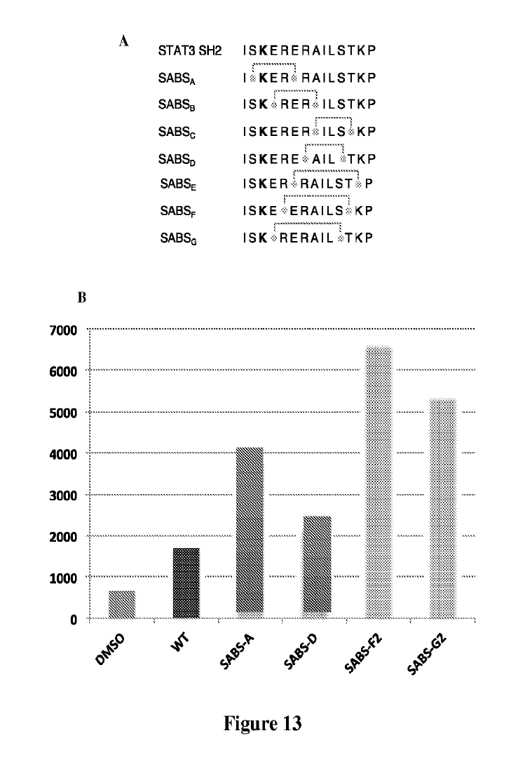

FIG. 13A showed additional exemplified stabilized sequences. FIG. 13B shows the cell penetration activities of the exemplified peptides. Specifically, Jurkat cells were incubated for 3 h at 37° C. with 5 μM fluorescently stapled sequences, compared to wildtype STAT3 SH2 peptide and DMSO as negative control.

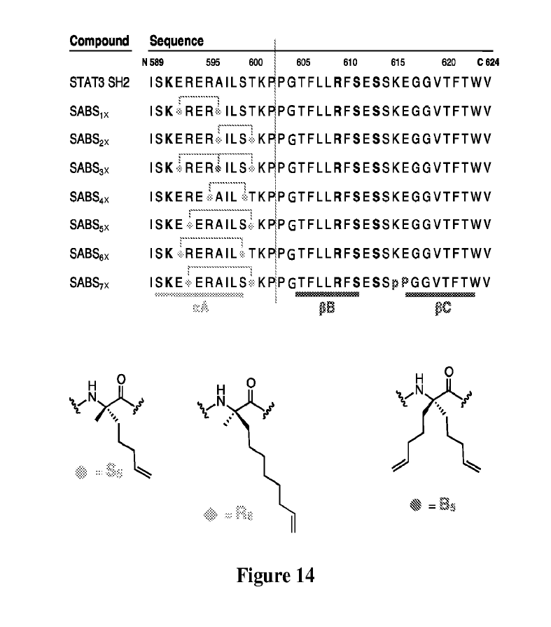

FIG. 14 shows additional exemplified stabilized sequences of the combined alpha helix and beta hairpin motif.

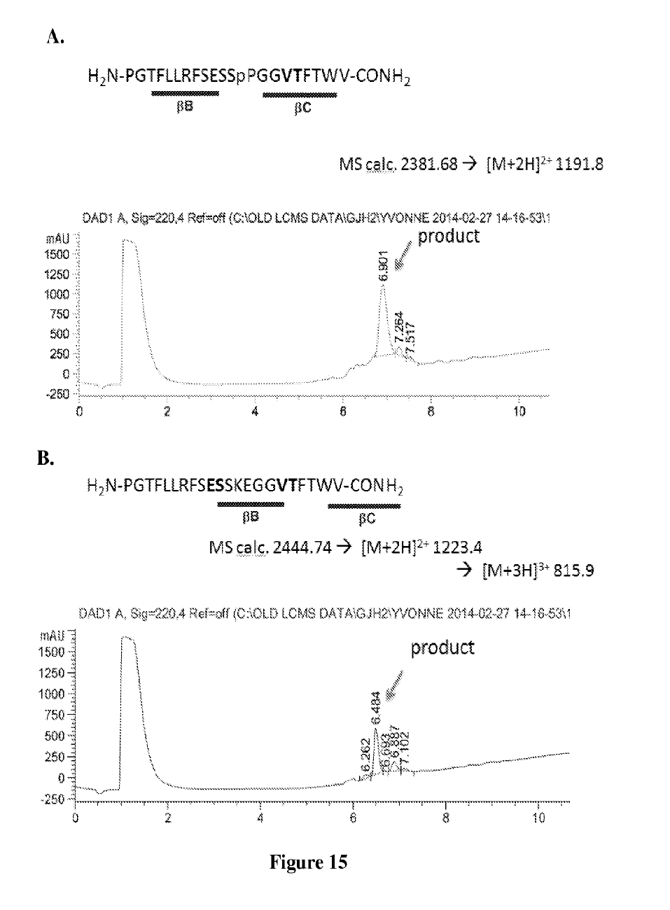

FIG. 15A and FIG. 15B shows two variants of beta-hairpin motif of STAT3 SH2 (from residues 624-603). Both variants were confirmed with LCMS. The bolded amino acid pair “ES” indicates the pseudoproline dipeptides ES, i.e. Fmoc-Glu(OtBu)-Ser(psi(Me,Me)pro)-OH. The bolded amino acid pair “VT” indicates the pseudoproline dipeptides VT, i.e. Fmoc-Val-Thr(psi(Me,Me)pro)-OH. The term “pP” stands for the D-Pro-L-Pro dipeptide.

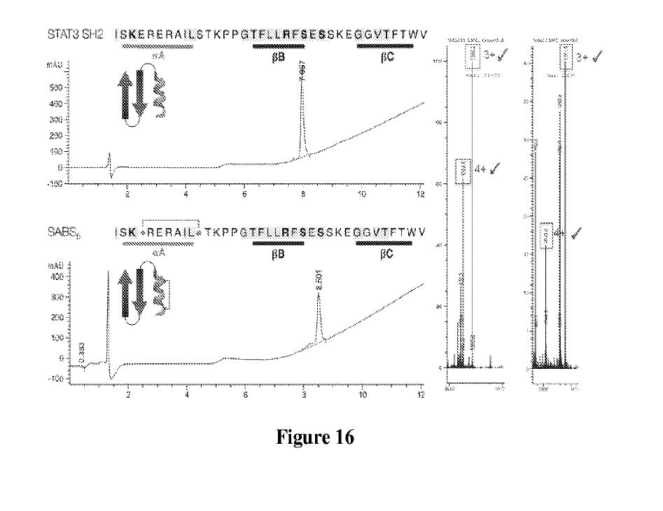

FIG. 16 shows preparation of the stabilized sequence having both the alpha-helical and the beta-hairpin motifs. The synthesized peptides are confirmed by LCMS. The synthesis was carried out with COMU (4 eq), each amino acid in the sequences (4 eq), N,N-Diisopropylethylamine (DIPEA) (8 eq), 1 h in N-methylpyrrolidone (NMP). The structure of COMU is as follows:

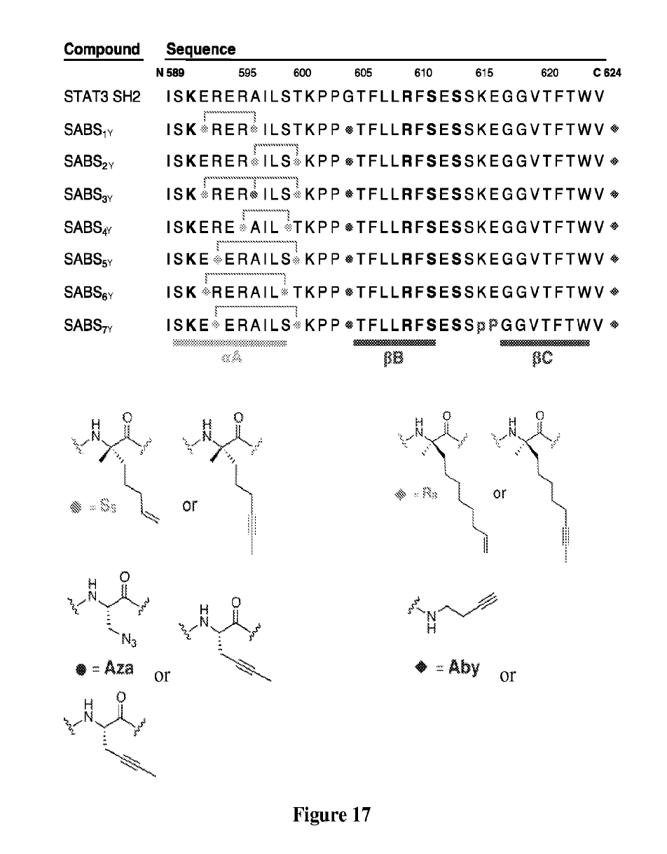

FIG. 17 shows exemplified polypeptides having both the alpha-helix and beta-hairpin stabilized by either the alkenylene cross-linker or alkynylene cross-linker generated from a RCM reaction or click chemistry reaction.

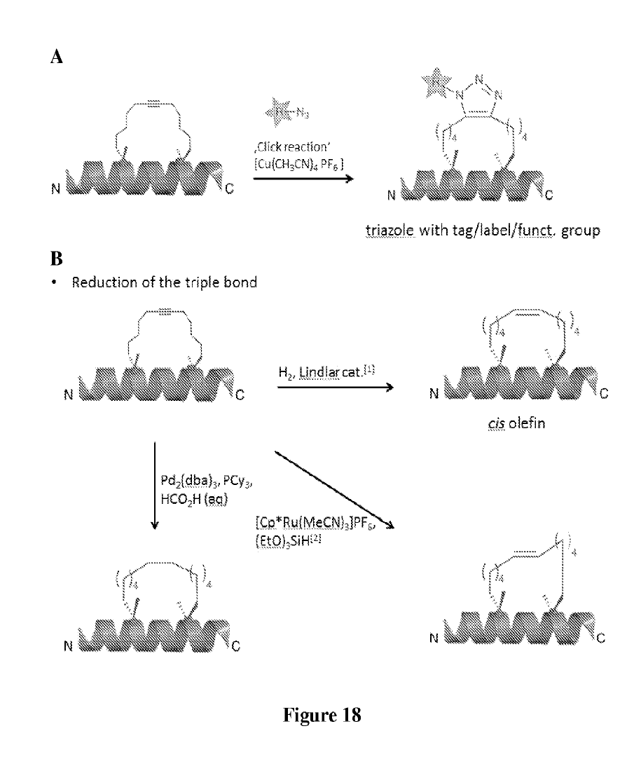

FIG. 18 shows exemplified post-RCM modification of the alkyne portion of the cross-linker in the polypeptide. FIG. 18A shows the click chemistry reaction of an optionally substituted azide with the alkyne portion of the cross-linker. FIG. 18B shows reduction of the alkyne portion of the cross-linker.

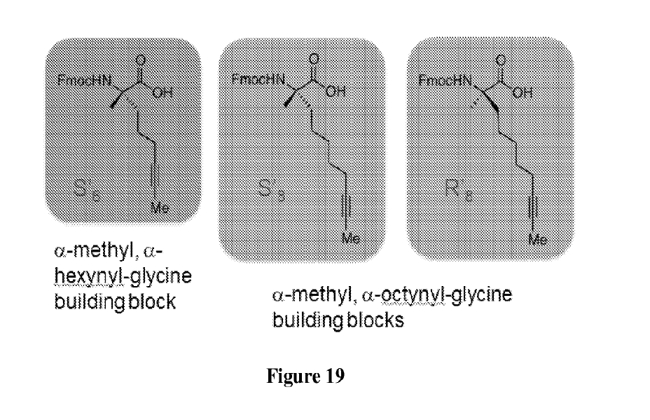

FIG. 19 shows exemplified compounds of Formula (AA).

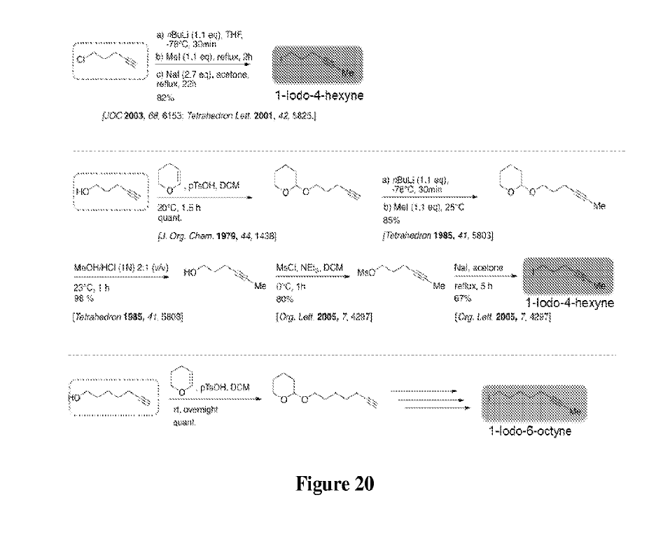

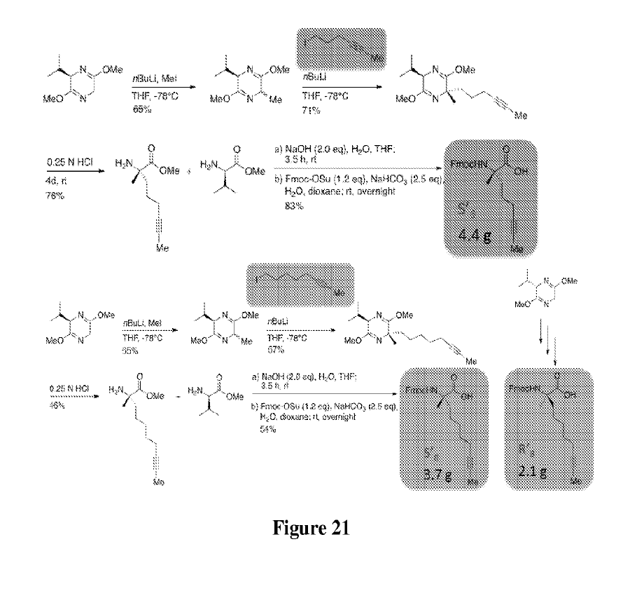

FIGS. 20-21 shows synthesis of exemplified compounds of Formula (AA).

The synthetic strategies are applicable to compounds of Formula (AA) with different tether lengths (i.e. length of L₁) by introducing the appropriate iodoalkyne (J. Org. Chem., 2003, 68, 6153; Tet. Lett., 2001, 42, 5825; Angew. Chem. Int. Ed. 1981, 20, 798-799; J. Org. Chem., 1979, 44, 1438; and Tetrahedron, 1985, 41, 5803; Org. Lett. 2005, 7, 4297.

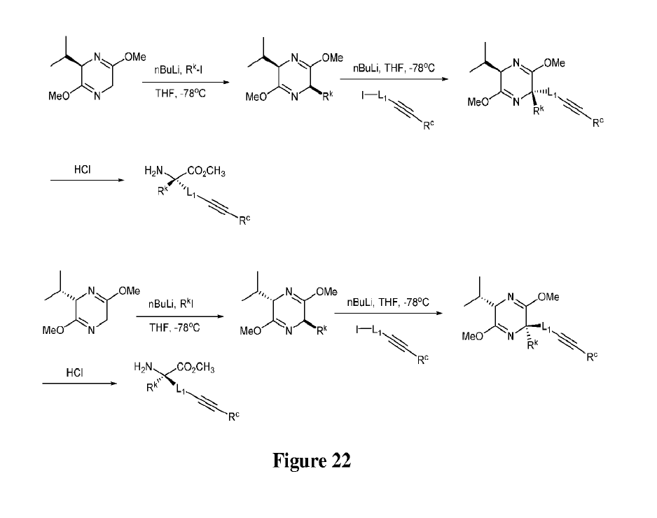

FIG. 22 provides a general synthetic scheme to prepare a compound of Formula (AA).

### DETAILED DESCRIPTION OF CERTAIN EMBODIMENTS OF THE INVENTION

The invention provides polypeptides (e.g. STAT polypeptides) comprising a stabilized alpha helix. The invention also provides polypeptides with more than one stabilized structural motif. In certain embodiments, the invention provides polypeptides comprising a stabilized alpha helix and an additional stabilized non-alpha helix motif (e.g., beta sheet or beta hairpin). In certain embodiments, the invention provides polypeptides comprising a stabilized alpha helix and a stabilized beta-hairpin (stabilized α,β-motif).

The stabilized polypeptides of the invention bear advantages such as potential high specificity, high potency in vitro and in vivo, a proteolytic stability, a generally favorable toxicity profile, as well as efficient entry to cells and access to intracellular targets.

In certain embodiments, the provided polypeptides are capable of binding a target and/or disrupting native or aberrant protein/protein interactions. In certain embodiments, the provided polypeptides are capable of disrupting STAT protein homodimerization.

In certain embodiments, the polypeptide is an oncoprotein or a derivative thereof. In certain embodiments, the oncoprotein is a STAT protein or a derivative thereof. In certain embodiments, the polypeptide is a STAT3 protein or derivative thereof. In certain embodiments, the provided STAT3 polypeptide comprising a stabilized alpha helix and a stabilized beta-hairpin is a cell-penetrating stabilized miniature protein that binds at the site of the phosphotyrosine of the STAT3 protein and inhibits STAT3 dimerization, thereby inhibiting STAT3 signaling and leading to the induction of apotosis. Therefore, the provided polypeptides are useful in the treatment of proliferative diseases such as cancer (e.g., breast cancer, lung cancer, kidney cancer, prostate cancer, or ovarian cancer), inflammatory diseases, autoimmune diseases, benign neoplams, etc.

In certain embodiments, the stabilized alpha helix of the provided polypeptide comprises at least two cross-linked amino acids. In certain embodiments, the stabilized alpha helix of the provided polypeptide has one cross-linker. In certain embodiments, the stabilized alpha helix of the provided polypeptide has more than one cross-linker. In certain embodiments, the cross-linker in the alpha helix is formed by a ring closing metathesis (RCM) reaction or click chemistry reaction. In certain embodiments, the cross-linker of the alpha helix is a hydrocarbon cross-linker. In other embodiments, the cross-linker of the alpha helix includes a heteroatom.

In certain embodiments, the stabilized beta-hairpin of the provided polypeptide comprises at least two cross-linked amino acids. In certain embodiments, the stabilized beta-hairpin of the provided polypeptide has one cross-linker. In certain embodiments, the stabilized beta-hairpin of the provided polypeptide has more than one cross-linker. In certain embodiments, the cross-linker in the beta-hairpin is formed by an RCM reaction or a click chemistry reaction. In certain embodiments, the cross-linker is an optionally substituted alkenylene. In certain embodiments, the cross-linker is an alkynylene. In certain embodiments, the cross-linker is an optionally substituted heteroarylene. In certain embodiments, the cross-linker is an optionally substituted five-membered heteroarylene.

In certain embodiments, the stabilized alpha helix of the provided polypeptide comprises at least one staple and/or at least one stitch. In certain embodiments, the staples and/or stitches in the stabilized alpha helix of the provided polypeptide are formed by an RCM reaction.

As generally used herein, the locations of the two cross-linked amino acids in the provided polypeptides are indicated as i and i+3, i and i+4, i and i+6, i and i+7, i and i+20, i and i+21, or i and i+22 in the polypeptide. The numerical value in the location indicator “i+numerical value” shows how many amino acids apart between the two cross-linked amino acids. In certain embodiments, the cross-linked amino acids in the stabilized alpha helix are at the i and i+3, i and i+4, i and i+6, i and i+7, or i and i+8 positions. In certain embodiments, stapling may occur at the i,i+3 positions, i,i+4 positions, and/or i,i+7 positions. In certain embodiments, the cross-linked amino acids in the stabilized alpha helix are at the i and i+4, or i and i+7 positions. In certain embodiments, the cross-linked amino acids in the stabilized alpha helix are at the i and i+4 positions. In certain embodiments, the cross-linked amino acids in the stabilized alpha helix are at the i and i+7 position. In certain embodiments, stitching may occur at the i,i+4+4 positions, the i,i+3+4 positions, the i,i+3+7 positions, or the i,i+4+7 positions. In certain embodiments, the cross-linked amino acids in the beta-hairpin are at i and i+20, i and i+21, i and i+22 positions.

In certain embodiments, the provided polypeptide comprises a stabilized alpha helix with one cross-linker and a stabilized beta-hairpin with one cross-linker. In certain embodiments, the provided polypeptide comprises a stabilized alpha helix with one stapled cross-linker formed by an RCM reaction and a stabilized beta-hairpin with one cross-linker formed by a click chemistry reaction. In certain embodiments, the provided polypeptide is a STAT peptide comprises a stabilized alpha helix with one stapled cross-linker formed by an RCM reaction and a stabilized beta-hairpin with one cross-linker formed by a click chemistry reaction. In certain embodiments, the provided polypeptide is a STAT3 peptide comprises a stabilized alpha helix with one stapled cross-linker formed by an RCM reaction and a stabilized beta-hairpin with one cross-linker formed by a click chemistry reaction (e.g. WO2010/033617). In certain embodiments, the provided polypeptide is derived from a STAT3 SH2 peptide (e.g., ISKERERAILSTKPPGTFLLRFSESSKEGGVTFTWV) or a derivative comprises a stabilized alpha helix with one stapled cross-linker formed by an RCM reaction and a stabilized beta-hairpin with one cross-linker formed by a click chemistry reaction.

In certain embodiments, the provided polypeptide is a STAT peptide or a derivative thereof comprising a stabilized alpha helix with at least one cross-linker. In certain embodiments, the cross-linker is a staple formed by an RCM reaction. In certain embodiments, the cross-linker is a stitch formed by an RCM reaction. In certain embodiments, the STAT polypeptide or a derivative thereof is a STAT3 peptide or a derivative thereof. In certain embodiments, the the STAT polypeptide or a derivative thereof is a STAT3 SH2 peptide (e.g., ISKERERAILSTKPPGTFLLRFSESSKEGGVTFTWV) or a derivative thereof.

As generally used herein, a STAT peptide refers to any member of the STAT (signal transducer and activator of transcription) family of proteins or mutants thereof. STAT family proteins include, but are not limited to, STAT1, STAT2, STAT3, STAT4, STAT5 (STAT5A and STAT5B), and STAT6. In certain embodiments, a STAT peptide is a mutant STAT. In certain embodiments, a STAT peptide is a substantially similar or a homologous form of the STAT family proteins. In certain embodiments, a STAT peptide is a substantially similar or a homologous form of a mutant STAT. In certain embodiments, a STAT peptide is STAT3 peptide or homologous form or mutant thereof.

As generally used herein, a polypeptide derivative thereof refers to a polypeptide produced from a wild type polypeptide either directly or by modification or partial substitution of one or more amino acids with one or more natural or unnatural amino acids. In certain embodiments, the polypeptide derivative is a STAT polypeptide derivative. In certain embodiments, the polypeptide derivative is a STAT3 polypeptide derivative. In certain embodiments, the polypeptide derivative is a STAT3 SH2 polypeptide derivative. In certain embodiments, the STAT polypeptide derivative is formed by partial substitution with one or more natural amino acids and one or more unnatural amino acids. In certain embodiments, the STAT3 polypeptide derivative is formed by partial substitution with one or more natural amino acids and one or more unnatural amino acids. In certain embodiments, the STAT3 SH2 polypeptide derivative is formed by partial substitution with one or more natural amino acids and one or more unnatural amino acids. In certain embodiments, the STAT3 SH2 polypeptide derivative is formed by substitution of K615 and E616 with D-Pro-L-Pro. In certain embodiments, the STAT3 SH2 polypeptide derivative is formed by partial substitution of S614 and K615 with L-Pro-D-Pro. In certain embodiments, the STAT3 SH2 polypeptide derivative is or is derived from ISKERERAILSTKPPGTFLLRFSESSpPGGVTFTWV (where p denotes D-Pro). In certain embodiments, the STAT3 SH2 polypeptide derivative is or is derived from ISKERERAILSTKPPGTFLLRFSESPpEGGVTFTWV (where “p” denotes D-Pro).

The present invention further provides stabilized polypeptide precursors of Formula (I):

wherein:

each instance of K, L₁, L₂, and M, is, independently, a bond, cyclic or acyclic, branched or unbranched, substituted or unsubstituted alkylene; cyclic or acyclic, branched or unbranched, substituted or unsubstituted alkenylene; cyclic or acyclic, branched or unbranched, substituted or unsubstituted alkynylene; cyclic or acyclic, branched or unbranched, substituted or unsubstituted heteroalkylene; cyclic or acyclic, branched or unbranched, substituted or unsubstituted heteroalkenylene; cyclic or acyclic, branched or unbranched, substituted or unsubstituted heteroalkynylene; substituted or unsubstituted arylene; substituted or unsubstituted heteroarylene; or substituted or unsubstituted acylene;

each instance of R^{a} is, independently, hydrogen; cyclic or acyclic, branched or unbranched, substituted or unsubstituted aliphatic; cyclic or acyclic, branched or unbranched, substituted or unsubstituted heteroaliphatic; substituted or unsubstituted aryl; substituted or unsubstituted heteroaryl; cyclic or acyclic, substituted or unsubstituted acyl; or R^{a} is a suitable amino protecting group;

each instance of R^{b} is, independently, a suitable amino acid side chain; hydrogen; cyclic or acyclic, branched or unbranched, substituted or unsubstituted aliphatic; cyclic or acyclic, branched or unbranched, substituted or unsubstituted heteroaliphatic; substituted or unsubstituted aryl; substituted or unsubstituted heteroaryl; cyclic or acyclic, substituted or unsubstituted acyl; substituted or unsubstituted hydroxyl; substituted or unsubstituted thiol; substituted or unsubstituted amino; cyano; isocyano; halo; or nitro;

each instance of R^{c}, is, independently, hydrogen; cyclic or acyclic, branched or unbranched, substituted or unsubstituted aliphatic; cyclic or acyclic, branched or unbranched, substituted or unsubstituted heteroaliphatic; substituted or unsubstituted aryl; substituted or unsubstituted heteroaryl; cyclic or acyclic, substituted or unsubstituted acyl; substituted or unsubstituted hydroxyl; substituted or unsubstituted thiol; substituted or unsubstituted amino; cyano; isocyano; halo; or nitro;

each instance of R^{e} is, independently, —R^{E}, —OR^{E}, —N(R^{E})₂, or —SR^{E}, wherein each instance of R^{E} is, independently, hydrogen, cyclic or acyclic, branched or unbranched, substituted or unsubstituted aliphatic; cyclic or acyclic, branched or unbranched, substituted or unsubstituted heteroaliphatic; substituted or unsubstituted aryl; substituted or unsubstituted heteroaryl; substituted or unsubstituted acyl; a resin; a suitable hydroxyl, amino or thiol protecting group; or two R^{E} groups together form a substituted or unsubstituted 5- to 6-membered heterocyclic or heteroaromatic ring;

each instance of R^{f} is, independently, hydrogen, cyclic or acyclic, branched or unbranched, substituted or unsubstituted aliphatic; cyclic or acyclic, branched or unbranched, substituted or unsubstituted heteroaliphatic; substituted or unsubstituted aryl; substituted or unsubstituted heteroaryl; substituted or unsubstituted acyl; a resin; a suitable amino protecting group; a label optionally joined by a linker, wherein the linker is selected from cyclic or acyclic, branched or unbranched, substituted or unsubstituted alkylene; cyclic or acyclic, branched or unbranched, substituted or unsubstituted alkenylene; cyclic or acyclic, branched or unbranched, substituted or unsubstituted alkynylene; cyclic or acyclic, branched or unbranched, substituted or unsubstituted heteroalkylene; cyclic or acyclic, branched or unbranched, substituted or unsubstituted heteroalkenylene; cyclic or acyclic, branched or unbranched, substituted or unsubstituted heteroalkynylene; substituted or unsubstituted arylene; substituted or unsubstituted heteroarylene; or substituted or unsubstituted acylene; or R^{f} and R^{a} together form a substituted or unsubstituted 5- to 6-membered heterocyclic or heteroaromatic ring;

each of A¹ and A² is independently selected from the group consisting of a leaving group (LG), —SH, —OH, —NH₂, —NH—NH₂, —N₃, —O—NH₂, —C(═O)R^{X1},

R^{X1} is hydrogen, a leaving group, or —OR^{X2}, wherein R^{X2} is hydrogen; optionally substituted alkyl; optionally substituted alkyl; optionally substituted alkenyl; optionally substituted alkynyl; optionally substituted carbocyclyl; optionally substituted heterocyclyl; optionally substituted aryl; optionally substituted heteroaryl: an oxygen protecting group;

Leaving group (LG) is —Br, —I, —Cl, —O(C═O)R^{LG}, or —O(SO)₂R^{LG}, wherein R^{LG} is optionally substituted alkyl, optionally substituted aryl, or optionally substituted heteroaryl;

W is O, S, or NR^{W1};

R^{W1} is hydrogen, optionally substituted alkyl; optionally substituted alkenyl; optionally substituted alkynyl; optionally substituted carbocyclyl; optionally substituted heterocyclyl; optionally substituted aryl; optionally substituted heteroaryl; or a nitrogen protecting group; and

R^{W2} is hydrogen, optionally substituted alkyl; optionally substituted alkenyl; optionally substituted alkynyl; optionally substituted carbocyclyl; optionally substituted heterocyclyl; optionally substituted aryl; optionally substituted heteroaryl, or two R^{W2} groups are joined to form a optionally substituted cyclic moiety;

each instance of X_{AA} is, independently, a natural or unnatural amino acid;

each instance of x is, independently, an integer between 0 to 3;

y is an integer between 2 to 8;

z1 and z2 is, independently, an integer between 2 to 30;

j is, independently, an integer between 1 to 10;

each instance of s and t is, independently, an integer between 0 and 100; and

wherein corresponds to a double or triple bond.

In some embodiments, the provided stabilized polypeptide precursors of Formula (I) undergo RCM and/or click chemistry reaction to form the inventive stabilized polypeptides.

In some embodiments, the stabilized polypeptide formed from Formula (I) is one of the following formulae:

wherein:

each instance of K, L₁, L₂, and M, is, independently, a bond, cyclic or acyclic, branched or unbranched, substituted or unsubstituted alkylene; cyclic or acyclic, branched or unbranched, substituted or unsubstituted alkenylene; cyclic or acyclic, branched or unbranched, substituted or unsubstituted alkynylene; cyclic or acyclic, branched or unbranched, substituted or unsubstituted heteroalkylene; cyclic or acyclic, branched or unbranched, substituted or unsubstituted heteroalkenylene; cyclic or acyclic, branched or unbranched, substituted or unsubstituted heteroalkynylene; substituted or unsubstituted arylene; substituted or unsubstituted heteroarylene; or substituted or unsubstituted acylene;

each instance of R^{a} is, independently, hydrogen; cyclic or acyclic, branched or unbranched, substituted or unsubstituted aliphatic; cyclic or acyclic, branched or unbranched, substituted or unsubstituted heteroaliphatic; substituted or unsubstituted aryl; substituted or unsubstituted heteroaryl; cyclic or acyclic, substituted or unsubstituted acyl; or R^{a} is a suitable amino protecting group:

each instance of R^{b} is, independently, a suitable amino acid side chain; hydrogen; cyclic or acyclic, branched or unbranched, substituted or unsubstituted aliphatic; cyclic or acyclic, branched or unbranched, substituted or unsubstituted heteroaliphatic; substituted or unsubstituted aryl; substituted or unsubstituted heteroaryl; cyclic or acyclic, substituted or unsubstituted acyl; substituted or unsubstituted hydroxyl; substituted or unsubstituted thiol; substituted or unsubstituted amino; cyano; isocyano; halo; or nitro;

each instance of R^{c}, is, independently, hydrogen; cyclic or acyclic, branched or unbranched, substituted or unsubstituted aliphatic; cyclic or acyclic, branched or unbranched, substituted or unsubstituted heteroaliphatic; substituted or unsubstituted aryl; substituted or unsubstituted heteroaryl; cyclic or acyclic, substituted or unsubstituted acyl; substituted or unsubstituted hydroxyl; substituted or unsubstituted thiol; substituted or unsubstituted amino; cyano; isocyano; halo; or nitro;

each instance of R^{e} is, independently, —R^{E}, —OR^{E}, —N(R^{E})₂, or —SR^{E}, wherein each instance of R^{E} is, independently, hydrogen, cyclic or acyclic, branched or unbranched, substituted or unsubstituted aliphatic; cyclic or acyclic, branched or unbranched, substituted or unsubstituted heteroaliphatic; substituted or unsubstituted aryl; substituted or unsubstituted heteroaryl; substituted or unsubstituted acyl; a resin; a suitable hydroxyl, amino, or thiol protecting group; or two R^{E} groups together form a substituted or unsubstituted 5- to 6-membered heterocyclic or heteroaromatic ring;

each instance of R^{f} is, independently, hydrogen; cyclic or acyclic, branched or unbranched, substituted or unsubstituted aliphatic; cyclic or acyclic, branched or unbranched, substituted or unsubstituted heteroaliphatic; substituted or unsubstituted aryl; substituted or unsubstituted heteroaryl; substituted or unsubstituted acyl; a resin: a suitable amino protecting group; a label optionally joined by a linker, wherein the linker is selected from cyclic or acyclic, branched or unbranched, substituted or unsubstituted alkylene; cyclic or acyclic, branched or unbranched, substituted or unsubstituted alkenylene; cyclic or acyclic, branched or unbranched, substituted or unsubstituted alkynylene; cyclic or acyclic, branched or unbranched, substituted or unsubstituted heteroalkylene; cyclic or acyclic, branched or unbranched, substituted or unsubstituted heteroalkenylene; cyclic or acyclic, branched or unbranched, substituted or unsubstituted heteroalkynylene; substituted or unsubstituted arylene; substituted or unsubstituted heteroarylene; or substituted or unsubstituted acylene; or R^{f} and R^{a} together form a substituted or unsubstituted 5- to 6-membered heterocyclic or heteroaromatic ring;

R^{KL} is hydrogen: cyclic or acyclic, branched or unbranched, substituted or unsubstituted aliphatic; cyclic or acyclic, branched or unbranched, substituted or unsubstituted heteroaliphatic; substituted or unsubstituted aryl; substituted or unsubstituted heteroaryl; substituted or unsubstituted acyl; substituted or unsubstituted hydroxyl; substituted or unsubstituted thiol; substituted or unsubstituted amino; azido; cyano; isocyano; halo; nitro;

or two adjacent R^{KL} groups are joined to form a substituted or unsubstituted 5- to 8-membered cycloaliphatic ring; substituted or unsubstituted 5- to 8-membered cycloheteroaliphatic ring; substituted or unsubstituted aryl ring; or substituted or unsubstituted heteroaryl ring; two adjacent R^{KL} groups are joined to form a substituted or unsubstituted 5- to 8-membered cycloaliphatic ring; substituted or unsubstituted 5- to 8-membered cycloheteroaliphatic ring; substituted or unsubstituted aryl ring; or substituted or unsubstituted heteroaryl ring; or two adjacent R^{LM} groups are joined to form a substituted or unsubstituted 5- to 8-membered cycloaliphatic ring; substituted or unsubstituted 5- to 8-membered cycloheteroaliphatic ring; substituted or unsubstituted aryl ring; or substituted or unsubstituted heteroaryl ring;

Q is —NH—, —NH—NH—, —O—NH—, —NH—O—, —S—, or —O—;

W is O, S, or NR^{W1};

R^{W2} is hydrogen, optionally substituted alkyl; optionally substituted alkenyl; optionally substituted alkynyl; optionally substituted carbocyclyl; optionally substituted heterocyclyl; optionally substituted aryl; optionally substituted heteroaryl; or a nitrogen protecting group; and

R^{W2} is hydrogen, optionally substituted alkyl; optionally substituted alkenyl; optionally substituted alkynyl; optionally substituted carbocyclyl; optionally substituted heterocyclyl; optionally substituted aryl; optionally substituted heteroaryl, or two R^{W2} groups are joined to form an optionally substituted cyclic moiety;

each instance of X_{AA} is, independently, a natural or unnatural amino acid;

each instance of x is, independently, an integer between 0 to 3;

each instance of y is, independently, an integer between 2 to 8;

each instance of z1 and z2 is, independently, an integer between 2 to 30;

each instance of j is, independently, an integer between 1 to 10;

each instance of s and t is, independently, an integer between 0 and 100;

each instance of v is, independently, an integer between 0 to 4; and

corresponds to a single, double, or triple bond.

In some embodiments, the invention provides a stabilized polypeptide precursor of Formula (II):

wherein:

each instance of K, L₁, L₂, and M, is, independently, a bond, cyclic or acyclic, branched or unbranched, substituted or unsubstituted alkylene; cyclic or acyclic, branched or unbranched, substituted or unsubstituted alkenylene; cyclic or acyclic, branched or unbranched, substituted or unsubstituted alkynylene; cyclic or acyclic, branched or unbranched, substituted or unsubstituted heteroalkylene; cyclic or acyclic, branched or unbranched, substituted or unsubstituted heteroalkenylene; cyclic or acyclic, branched or unbranched, substituted or unsubstituted heteroalkynylene; substituted or unsubstituted arylene; substituted or unsubstituted heteroarylene; or substituted or unsubstituted acylene;

each instance of R^{a} is, independently, hydrogen; cyclic or acyclic, branched or unbranched, substituted or unsubstituted aliphatic; cyclic or acyclic, branched or unbranched, substituted or unsubstituted heteroaliphatic; substituted or unsubstituted aryl; substituted or unsubstituted heteroaryl; cyclic or acyclic, substituted or unsubstituted acyl; or R^{a} is a suitable amino protecting group;

each instance of R^{b} is, independently, a suitable amino acid side chain; hydrogen; cyclic or acyclic, branched or unbranched, substituted or unsubstituted aliphatic; cyclic or acyclic, branched or unbranched, substituted or unsubstituted heteroaliphatic; substituted or unsubstituted aryl; substituted or unsubstituted heteroaryl; cyclic or acyclic, substituted or unsubstituted acyl; substituted or unsubstituted hydroxyl; substituted or unsubstituted thiol; substituted or unsubstituted amino; cyano; isocyano; halo; or nitro;

each instance of R^{c}, is, independently, hydrogen; cyclic or acyclic, branched or unbranched, substituted or unsubstituted aliphatic; cyclic or acyclic, branched or unbranched, substituted or unsubstituted heteroaliphatic; substituted or unsubstituted aryl; substituted or unsubstituted heteroaryl; cyclic or acyclic, substituted or unsubstituted acyl; substituted or unsubstituted hydroxyl; substituted or unsubstituted thiol; substituted or unsubstituted amino; cyano; isocyano; halo; or nitro;

each instance of R^{e} is, independently, —R^{E}, —OR^{E}, —N(R^{E})₂, or —SR^{E}, wherein each instance of R^{E} is, independently, hydrogen, cyclic or acyclic, branched or unbranched, substituted or unsubstituted aliphatic; cyclic or acyclic, branched or unbranched, substituted or unsubstituted heteroaliphatic; substituted or unsubstituted aryl; substituted or unsubstituted heteroaryl; substituted or unsubstituted acyl; a resin; a suitable hydroxyl, amino or thiol protecting group; or two R^{E} groups together form a substituted or unsubstituted 5- to 6-membered heterocyclic or heteroaromatic ring;

each instance of R^{f} is, independently, hydrogen, cyclic or acyclic, branched or unbranched, substituted or unsubstituted aliphatic; cyclic or acyclic, branched or unbranched, substituted or unsubstituted heteroaliphatic; substituted or unsubstituted aryl; substituted or unsubstituted heteroaryl; substituted or unsubstituted acyl; a resin; a suitable amino protecting group; a label optionally joined by a linker, wherein the linker is selected from cyclic or acyclic, branched or unbranched, substituted or unsubstituted alkylene; cyclic or acyclic, branched or unbranched, substituted or unsubstituted alkenylene; cyclic or acyclic, branched or unbranched, substituted or unsubstituted alkynylene; cyclic or acyclic, branched or unbranched, substituted or unsubstituted heteroalkylene; cyclic or acyclic, branched or unbranched, substituted or unsubstituted heteroalkenylene; cyclic or acyclic, branched or unbranched, substituted or unsubstituted heteroalkynylene; substituted or unsubstituted arylene; substituted or unsubstituted heteroarylene; or substituted or unsubstituted acylene; or R^{f} and R^{a} together form a substituted or unsubstituted 5- to 6-membered heterocyclic or heteroaromatic ring;

each of A¹ and A² is independently selected from the group consisting of a leaving group (LG), —SH, —OH, —NH₂, —NH—NH₂, —N₃, —O—NH₂, —C(═O)R^{X1},

R^{X1} is hydrogen, a leaving group, or —OR^{X2}, wherein R^{X2} is hydrogen; optionally substituted alkyl; optionally substituted alkyl; optionally substituted alkenyl; optionally substituted alkynyl; optionally substituted carbocyclyl; optionally substituted heterocyclyl; optionally substituted aryl; optionally substituted heteroaryl; an oxygen protecting group;

Leaving group (LG) is —Br, —I, —Cl, —O(C═O)R^{LG}, or —O(SO)₂R^{LG}, wherein R^{LG} is optionally substituted alkyl, optionally substituted aryl, or optionally substituted heteroaryl;

W is O, S, or NR^{W1};

R^{W1} is hydrogen, optionally substituted alkyl; optionally substituted alkenyl; optionally substituted alkynyl; optionally substituted carbocyclyl; optionally substituted heterocyclyl; optionally substituted aryl; optionally substituted heteroaryl; or a nitrogen protecting group; and

R^{W2} is hydrogen, optionally substituted alkyl; optionally substituted alkenyl; optionally substituted alkynyl; optionally substituted carbocyclyl; optionally substituted heterocyclyl; optionally substituted aryl; optionally substituted heteroaryl, or two R^{W2} groups are joined to form a optionally substituted cyclic moiety;

each instance of X_{AA} is, independently, a natural or unnatural amino acid;

each instance of x is, independently, an integer between 0 to 3;

each instance of y and z are, independently, an integer between 2 to 8;

each instance of z1 and z2 is, independently, an integer between 2 to 30;

j is, independently, an integer between 1 to 10;

p is an integer between 0 to 10;

each instance of s and t is, independently, an integer between 0 and 100; and

wherein corresponds to a double or triple bond.

In some embodiments, the stabilized polypeptide formed by RCM and/or click chemistry reaction from the precursor of Formula (II) is of Formula (III):

wherein:

each instance of K, L₁, L₂, and M, is, independently, a bond, cyclic or acyclic, branched or unbranched, substituted or unsubstituted alkylene; cyclic or acyclic, branched or unbranched, substituted or unsubstituted alkenylene; cyclic or acyclic, branched or unbranched, substituted or unsubstituted alkynylene; cyclic or acyclic, branched or unbranched, substituted or unsubstituted heteroalkylene; cyclic or acyclic, branched or unbranched, substituted or unsubstituted heteroalkenylene; cyclic or acyclic, branched or unbranched, substituted or unsubstituted heteroalkynylene; substituted or unsubstituted arylene; substituted or unsubstituted heteroarylene; or substituted or unsubstituted acylene;

each instance of R^{a} is, independently, hydrogen; cyclic or acyclic, branched or unbranched, substituted or unsubstituted aliphatic; cyclic or acyclic, branched or unbranched, substituted or unsubstituted heteroaliphatic; substituted or unsubstituted aryl; substituted or unsubstituted heteroaryl; cyclic or acyclic, substituted or unsubstituted acyl; or R^{a} is a suitable amino protecting group:

each instance of R^{b} is, independently, a suitable amino acid side chain; hydrogen; cyclic or acyclic, branched or unbranched, substituted or unsubstituted aliphatic; cyclic or acyclic, branched or unbranched, substituted or unsubstituted heteroaliphatic; substituted or unsubstituted aryl; substituted or unsubstituted heteroaryl; cyclic or acyclic, substituted or unsubstituted acyl; substituted or unsubstituted hydroxyl; substituted or unsubstituted thiol; substituted or unsubstituted amino; cyano; isocyano; halo; or nitro;

each instance of Re is, independently, —R^{E}, —OR^{E}, —N(R^{E})₂, or —SR^{E}, wherein each instance of R^{E} is, independently, hydrogen, cyclic or acyclic, branched or unbranched, substituted or unsubstituted aliphatic; cyclic or acyclic, branched or unbranched, substituted or unsubstituted heteroaliphatic; substituted or unsubstituted aryl; substituted or unsubstituted heteroaryl; substituted or unsubstituted acyl; a resin: a suitable hydroxyl, amino, or thiol protecting group; or two R^{E} groups together form a substituted or unsubstituted 5- to 6-membered heterocyclic or heteroaromatic ring;

each instance of R^{f} is, independently, hydrogen; cyclic or acyclic, branched or unbranched, substituted or unsubstituted aliphatic; cyclic or acyclic, branched or unbranched, substituted or unsubstituted heteroaliphatic; substituted or unsubstituted aryl; substituted or unsubstituted heteroaryl; substituted or unsubstituted acyl; a resin; a suitable amino protecting group; a label optionally joined by a linker, wherein the linker is selected from cyclic or acyclic, branched or unbranched, substituted or unsubstituted alkylene; cyclic or acyclic, branched or unbranched, substituted or unsubstituted alkenylene; cyclic or acyclic, branched or unbranched, substituted or unsubstituted alkynylene; cyclic or acyclic, branched or unbranched, substituted or unsubstituted heteroalkylene; cyclic or acyclic, branched or unbranched, substituted or unsubstituted heteroalkenylene; cyclic or acyclic, branched or unbranched, substituted or unsubstituted heteroalkynylene; substituted or unsubstituted arylene; substituted or unsubstituted heteroarylene; or substituted or unsubstituted acylene; or R^{f} and R^{a} together form a substituted or unsubstituted 5- to 6-membered heterocyclic or heteroaromatic ring;

each instance of R^{KL}, R^{LL}, and R^{LM}, is, independently, hydrogen; cyclic or acyclic, branched or unbranched, substituted or unsubstituted aliphatic; cyclic or acyclic, branched or unbranched, substituted or unsubstituted heteroaliphatic; substituted or unsubstituted aryl; substituted or unsubstituted heteroaryl; substituted or unsubstituted acyl; substituted or unsubstituted hydroxyl; substituted or unsubstituted thiol; substituted or unsubstituted amino; azido; cyano; isocyano; halo; nitro;

or two adjacent R^{KL} groups are joined to form a substituted or unsubstituted 5- to 8-membered cycloaliphatic ring; substituted or unsubstituted 5- to 8-membered cycloheteroaliphatic ring; substituted or unsubstituted aryl ring; or substituted or unsubstituted heteroaryl ring; two adjacent R^{KL} groups are joined to form a substituted or unsubstituted 5- to 8-membered cycloaliphatic ring; substituted or unsubstituted 5- to 8-membered cycloheteroaliphatic ring; substituted or unsubstituted aryl ring; or substituted or unsubstituted heteroaryl ring; or two adjacent R^{LM} groups are joined to form a substituted or unsubstituted 5- to 8-membered cycloaliphatic ring; substituted or unsubstituted 5- to 8-membered cycloheteroaliphatic ring; substituted or unsubstituted aryl ring; or substituted or unsubstituted heteroaryl ring;

Q is —NH—, —NH—NH—, —O—NH—, —NH—O—, —S—, or —O—;

W is O, S, or NR^{W1};

R^{W2} is hydrogen, optionally substituted alkyl; optionally substituted alkenyl; optionally substituted alkynyl; optionally substituted carbocyclyl; optionally substituted heterocyclyl; optionally substituted aryl; optionally substituted heteroaryl; or a nitrogen protecting group; and

R^{W2} is hydrogen, optionally substituted alkyl; optionally substituted alkenyl; optionally substituted alkynyl; optionally substituted carbocyclyl; optionally substituted heterocyclyl; optionally substituted aryl; optionally substituted heteroaryl, or two R^{W2} groups are joined to form an optionally substituted cyclic moiety;

each instance of X_{AA} is, independently, a natural or unnatural amino acid;

each instance of x is, independently, an integer between 0 to 3;

each instance of y and z is, independently, an integer between 2 to 8;

each instance of z1 and z2 is, independently, an integer between 2 to 30;

each instance of j is, independently, an integer between 1 to 10;

each instance of p is, independently, an integer between 0 to 10;

each instance of s and t is, independently, an integer between 0 and 100;

each instance of u, v, and q, is, independently, an integer between 0 to 4;

and wherein:

corresponds to a single, double, or triple bond.

As generally defined herein, R^{W2} is hydrogen, optionally substituted alkyl;

optionally substituted alkenyl; optionally substituted alkynyl; optionally substituted carbocyclyl; optionally substituted heterocyclyl; optionally substituted aryl; optionally substituted heteroaryl, or two R^{W2} groups are joined to form a optionally substituted cyclic moiety. In certain embodiments, R^{W2} is hydrogen. In certain embodiments, R^{W2} is halogen. In certain embodiments, R^{W2} is F. In certain embodiments, R^{W2} is Cl. In certain embodiments, R^{W2} is Br. In certain embodiments, R^{W2} is I. In certain embodiments, R^{W2} is optionally substituted alkyl. In certain embodiments, R^{W2} is optionally substituted C₁₋₆ alkyl. In certain embodiments, R^{W2} is unsubstituted C₁₋₆ alkyl (e.g. methyl or ethyl). In certain embodiments, R^{W2} is substituted C₁₋₆ alkyl (e.g. C₁₋₆ haloalkyl).

In certain embodiments, each of A¹ and A² is independently

### or —N₃.

In certain embodiments, each of A¹ and A² is independently

### or —N₃.

In certain embodiments, A is

In certain embodiments, A is

In certain embodiments, a provided polypeptide comprises a stabilized STAT peptide or a derivative thereof, or a precursor of a stabilized STAT peptide or a derivative thereof.

In certain embodiments, a provided polypeptide comprises a STAT3 peptide or a derivative thereof.

In certain embodiments, a provided polypeptide comprises a STAT3 SH2 peptide (ISKERERAILSTKPPGTFLLRFSESSKEGGVTFTWV) or a derivative thereof.

In certain embodiments, a provided polypeptide comprises a STAT3 SH2 peptide derivative that is derived from ISKERERAILSTKPPGTFLLRFSESSpPGGVTFTWV or ISKERERAILSTKPPGTFLLRFSESPpEGGVTFTWV.

In certain embodiments, corresponds to a double bond. In certain embodiments, corresponds to a triple bond.

In certain embodiments, all corresponds to a single bond, and u, v and q are, independently, 0, 1, 2, 3, or 4.

In certain embodiments, all corresponds to a double bond, u, v and q are, independently, 0, 1, or 2.

In certain embodiments, each instance of K, L₁, L₂, and M, independently, corresponds to a bond, cyclic or acyclic, branched or unbranched, substituted or unsubstituted C₁₋₂₀ alkylene; cyclic or acyclic, branched or unbranched, substituted or unsubstituted C₁₋₂₀ alkenylene; cyclic or acyclic, branched or unbranched, substituted or unsubstituted C₁₋₂₀ alkynylene; cyclic or acyclic, branched or unbranched, substituted or unsubstituted C₁₋₂₀ heteroalkylene; cyclic or acyclic, branched or unbranched, substituted or unsubstituted C₁₋₂₀ heteroalkenylene; cyclic or acyclic, branched or unbranched, substituted or unsubstituted C₁₋₂₀ heteroalkynylene; substituted or unsubstituted C₁₋₂₀ arylene; substituted or unsubstituted C₁₋₂₀ heteroarylene; or substituted or unsubstituted C₁₋₂₀ acylene; cyclic or acyclic, branched or unbranched, substituted or unsubstituted C₁₋₁₅ alkylene; cyclic or acyclic, branched or unbranched, substituted or unsubstituted C₁₋₁₅ alkenylene; cyclic or acyclic, branched or unbranched, substituted or unsubstituted C₁₋₁₅ alkynylene; cyclic or acyclic, branched or unbranched, substituted or unsubstituted C₁₋₁₅ heteroalkylene; cyclic or acyclic, branched or unbranched, substituted or unsubstituted C₁₋₁₅ heteroalkenylene; cyclic or acyclic, branched or unbranched, substituted or unsubstituted C₁₋₁₅ heteroalkynylene; substituted or unsubstituted C₁₋₁₅ arylene; substituted or unsubstituted C₁₋₁₅ heteroarylene; or substituted or unsubstituted C₁₋₁₀ acylene; cyclic or acyclic, branched or unbranched, substituted or unsubstituted C₁₋₁₀ alkylene; cyclic or acyclic, branched or unbranched, substituted or unsubstituted C₁₋₁₀ alkenylene; cyclic or acyclic, branched or unbranched, substituted or unsubstituted C₁₋₁₀ alkynylene; cyclic or acyclic, branched or unbranched, substituted or unsubstituted C₁₋₁₀ heteroalkylene; cyclic or acyclic, branched or unbranched, substituted or unsubstituted C₁₋₁₀ heteroalkenylene; cyclic or acyclic, branched or unbranched, substituted or unsubstituted C₁₋₁₀ heteroalkynylene; substituted or unsubstituted C₁₋₁₀ arylene; substituted or unsubstituted C₁₋₁₀ heteroarylene; or substituted or unsubstituted C₁₋₁₀ acylene; cyclic or acyclic, branched or unbranched, substituted or unsubstituted C₁₋₈ alkylene; cyclic or acyclic, branched or unbranched, substituted or unsubstituted C₁₋₈alkenylene; cyclic or acyclic, branched or unbranched, substituted or unsubstituted C₁₋₈ alkynylene; cyclic or acyclic, branched or unbranched, substituted or unsubstituted C₁₋₈ heteroalkylene; cyclic or acyclic, branched or unbranched, substituted or unsubstituted C₁₋₈ heteroalkenylene; cyclic or acyclic, branched or unbranched, substituted or unsubstituted C₁₋₈ heteroalkynylene; substituted or unsubstituted C₁₋₈ arylene; substituted or unsubstituted C₁₋₈ heteroarylene; or substituted or unsubstituted C₁₋₈ acylene; cyclic or acyclic, branched or unbranched, substituted or unsubstituted C₁₋₅ alkylene; cyclic or acyclic, branched or unbranched, substituted or unsubstituted C₁₋₅ alkenylene; cyclic or acyclic, branched or unbranched, substituted or unsubstituted C₁₋₅ alkynylene; cyclic or acyclic, branched or unbranched, substituted or unsubstituted C₁₋₅ heteroalkylene; cyclic or acyclic, branched or unbranched, substituted or unsubstituted C₁₋₅ heteroalkenylene; cyclic or acyclic, branched or unbranched, substituted or unsubstituted C₁₋₅ heteroalkynylene; substituted or unsubstituted C₁₋₅ arylene; substituted or unsubstituted C₁₋₅ heteroarylene; or substituted or unsubstituted C₁₋₅ acylene.

In certain embodiments, K is acyclic. In certain embodiments, K is unbranched. In certain embodiments, K is unsubstituted. In certain embodiments, K is a bond. In certain embodiments, K is not a bond.

In certain embodiments, M is acyclic. In certain embodiments, M is unbranched. In certain embodiments, M is unsubstituted. In certain embodiments, M is a bond. In certain embodiments, M is not a bond.

In certain embodiments, L₁ is acyclic. In certain embodiments, L₁ is unbranched. In certain embodiments, L₁ is unsubstituted. In certain embodiments, L₁ is a bond. In certain embodiments, L₁ is not a bond.

In certain embodiments, L₂ is acyclic. In certain embodiments, L₂ is unbranched. In certain embodiments, L₂ is unsubstituted. In certain embodiments, L₂ is a bond. In certain embodiments, L₂ is not a bond.

In certain embodiments, L₁ and L₂ are the same. In certain embodiments, L₁ and L₂ are different. In certain embodiments, when L₁ is a bond, L₂ is not a bond, or when L₂ is a bond, L₁ is not a bond. In certain embodiments, a polypeptide of any of the above formulae wherein L₁ and L₂ are both bonds is specifically excluded.

In certain embodiments, K and M are the same. In certain embodiments, K and M are different.

In certain embodiments, K and L₁ are the same. In certain embodiments, K and L₁ are different. In certain embodiments, K and L₂ are the same. In certain embodiments, K and L₂ are different.

In certain embodiments, M and L₁ are the same. In certain embodiments, M and L₁ are different. In certain embodiments, M and L₂ are the same. In certain embodiments, M and L₂ are different.

In certain embodiments, all of K, L₁, L₂, and M are the same. In certain embodiments, all of K, L₁, L₂, and M are different.

In certain embodiments, each instance of K, L₁, L₂, and M, independently, corresponds to the formulae: —(CH₂)_{g+1}—; —(CH₂)_{g}—S—(CH₂)_{g}—; —(CH₂)_{g}—(C═O)—S—(CH₂)_{g}—; —(CH₂)_{g}—O(CH₂)_{g}—; —(CH₂)_{g}—(C═O)—O—(CH₂)_{g}—; —(CH₂)_{g}—NH—(CH₂)_{g}—; —(CH₂)_{g}—(CO)—NH—(CH₂)_{g}—; —(CH₂)_{g}CH(CH₃)—O—(CH₂)_{g}—;

wherein each instance of g is, independently, 0 to 10, inclusive.

In certain embodiments, each instance of K, L₁, L₂, and M, independently, corresponds to the formulae —(CH₂)_{g+1}—, and g is 0, 1, 2, 3, 4, 5, or 6.

In certain embodiments, —[X_{AA}]— corresponds to the formulae:

wherein:
each instance of R and R′ are, independently, hydrogen, or a suitable amino acid side chain as defined herein, and R^{a} is as previously defined above and herein.

Suitable amino acid side chains include, but are not limited to, both natural and unnatural amino acid side chains as provided in Tables 1 to 3, and as described herein. In certain embodiments, each instance of X_{AA} is an alpha amino acid, corresponding to the formula (α). In certain embodiments, each instance of X_{AA} is a natural L-amino acid, as provided in Table 1. In certain embodiments, each instance of X_{AA} is, independently, a natural L-amino acid as provided in Table 1, or an unnatural D-amino acid as provided in Table 2.

The group R^{e} corresponds to the C-terminus of the peptide chain, and corresponds to the variables —R^{E}, —OR^{E}, —N(R^{E})₂, or —SR^{E}, wherein R^{E} is as defined above and herein. For example, if —[X_{AA}]— corresponds to an alpha amino acid of formula:

it follows that, in certain embodiments, —[X_{AA}]ₜ—R^{e} corresponds to the formulae:

wherein each instance of R^{E} is, independently, hydrogen; cyclic or acyclic, branched or unbranched, substituted or unsubstituted aliphatic; cyclic or acyclic, branched or unbranched, substituted or unsubstituted heteroaliphatic; substituted or unsubstituted aryl; substituted or unsubstituted heteroaryl; substituted or unsubstituted acyl; a resin; or a suitable hydroxyl, amino, or thiol protecting group; and two R^{E} groups taken together may optionally form a substituted or unsubstituted 5- to 6-membered heterocyclic or heteroaromatic ring.

In certain embodiments, R^{e} is —OR^{E}, and R^{E} is hydrogen, cyclic or acyclic, branched or unbranched, substituted or unsubstituted aliphatic; cyclic or acyclic, branched or unbranched, substituted or unsubstituted heteroaliphatic; substituted or unsubstituted aryl; substituted or unsubstituted heteroaryl; substituted or unsubstituted acyl; a resin: or a suitable hydroxyl protecting group.

In certain embodiments, R^{e} is —SR^{E}, and R^{E} is hydrogen, cyclic or acyclic, branched or unbranched, substituted or unsubstituted aliphatic; cyclic or acyclic, branched or unbranched, substituted or unsubstituted heteroaliphatic; substituted or unsubstituted aryl; substituted or unsubstituted heteroaryl; substituted or unsubstituted acyl; a resin; or a suitable thiol protecting group.

In certain embodiments, R is —N(R^{E})₂, and each instance of R^{E} is, independently, hydrogen, cyclic or acyclic, branched or unbranched, substituted or unsubstituted aliphatic; cyclic or acyclic, branched or unbranched, substituted or unsubstituted heteroaliphatic; substituted or unsubstituted aryl; substituted or unsubstituted heteroaryl; substituted or unsubstituted acyl; a resin; a suitable amino protecting group; or two R^{E} groups together form a substituted or unsubstituted 5- to 6-membered heterocyclic or heteroaromatic ring.

The group R^{f} corresponds to the N-terminus of the peptide chain. For example, if —[X_{AA}]— corresponds to an alpha amino acid of formula:

it follows that, in certain embodiments, R^{f}—[X_{AA}]— corresponds to the formulae:

wherein R and R′ are defined as above and herein; and
wherein R^{f} is hydrogen; cyclic or acyclic, branched or unbranched, substituted or unsubstituted aliphatic; cyclic or acyclic, branched or unbranched, substituted or unsubstituted heteroaliphatic; substituted or unsubstituted aryl; substituted or unsubstituted heteroaryl; substituted or unsubstituted acyl; a resin; a suitable amino protecting group; a label optionally joined by a linker, wherein the linker is selected from cyclic or acyclic, branched or unbranched, substituted or unsubstituted alkylene; cyclic or acyclic, branched or unbranched, substituted or unsubstituted alkenylene; cyclic or acyclic, branched or unbranched, substituted or unsubstituted alkynylene; cyclic or acyclic, branched or unbranched, substituted or unsubstituted heteroalkylene; cyclic or acyclic, branched or unbranched, substituted or unsubstituted heteroalkenylene; cyclic or acyclic, branched or unbranched, substituted or unsubstituted heteroalkynylene; substituted or unsubstituted arylene; substituted or unsubstituted heteroarylene; or substituted or unsubstituted acylene; or R^{f} and R^{a} together form a substituted or unsubstituted 5- to 6-membered heterocyclic or heteroaromatic ring.

In certain embodiments, R^{f} is hydrogen. In certain embodiments, R^{f} is C₁₋₆ alkyl. In certain embodiments, R^{f} is —CH₃. In certain embodiments, R^{f} is a suitable amino protecting group. In certain embodiments, R^{f} is -Boc. In certain embodiments, R^{f} is -Fmoc. In certain embodiments, R^{f} is acyl. In certain embodiments, R^{f} is —(C═O)CH₃.

In certain embodiments, R^{f} is a label optionally joined by a linker, wherein the linker is selected from cyclic or acyclic, branched or unbranched, substituted or unsubstituted alkylene; cyclic or acyclic, branched or unbranched, substituted or unsubstituted alkenylene; cyclic or acyclic, branched or unbranched, substituted or unsubstituted alkynylene; cyclic or acyclic, branched or unbranched, substituted or unsubstituted heteroalkylene; cyclic or acyclic, branched or unbranched, substituted or unsubstituted heteroalkenylene; cyclic or acyclic, branched or unbranched, substituted or unsubstituted heteroalkynylene; substituted or unsubstituted arylene; substituted or unsubstituted heteroarylene; or substituted or unsubstituted acylene.

Exemplary labels include, but are not limited to FITC and biotin:

In certain embodiments, the label is directly joined to the inventive polypeptide (i.e., through a bond).

In certain embodiments, the label is indirectly joined to the inventive polypeptide (i.e., through a linker).

In certain embodiments, the linker is a cyclic or acyclic, branched or unbranched, substituted or unsubstituted alkylene. In certain embodiments, the linker is a cyclic or acyclic, branched or unbranched, substituted or unsubstituted alkenylene. In certain embodiments, the linker is a cyclic or acyclic, branched or unbranched, substituted or unsubstituted alkynylene. In certain embodiments, the linker is a cyclic or acyclic, branched or unbranched, substituted or unsubstituted heteroalkylene. In certain embodiments, the linker is a cyclic or acyclic, branched or unbranched, substituted or unsubstituted heteroalkenylene. In certain embodiments, the linker is a cyclic or acyclic, branched or unbranched, substituted or unsubstituted heteroalkynylene. In certain embodiments, the linker is a substituted or unsubstituted arylene. In certain embodiments, the linker is a substituted or unsubstituted heteroarylene. In certain embodiments, the linker is a substituted or unsubstituted acylene.

For example, in certain embodiments, the linker is cyclic or acyclic, branched or unbranched, substituted or unsubstituted heteroalkylene selected from:

In certain embodiments, R^{a} is hydrogen. In certain embodiments, R^{a} is C₁₋₆ alkyl.

In certain embodiments, R^{a} is —CH₃. In certain embodiments, R^{a} is acyl. In certain embodiments, R^{a} is —(C═O)CH₃.

In certain embodiments, each instance of R^{b} is, independently, hydrogen or cyclic or acyclic, branched or unbranched, substituted or unsubstituted aliphatic. In certain embodiments, R^{b} is hydrogen or —CH₃. In certain embodiments, R^{b} is —CH₃.

In certain embodiments, each instance of R^{c}, is, independently, hydrogen; cyclic or acyclic, branched or unbranched, substituted or unsubstituted aliphatic; cyclic or acyclic, branched or unbranched, substituted or unsubstituted heteroaliphatic; substituted or unsubstituted aryl; substituted or unsubstituted heteroaryl. In certain embodiments, each instance of R^{c}, is, independently, hydrogen; or cyclic or acyclic, branched or unbranched, substituted or unsubstituted aliphatic. In certain embodiments, each instance of R^{c} is, independently, hydrogen or cyclic or acyclic, branched or unbranched, substituted or unsubstituted alkyl. In certain embodiments, R^{b} is hydrogen or —CH₃. In certain embodiments, each instance of R^{c} is hydrogen.

In certain embodiments, each instance of R^{KL}, R^{LL}, and R^{LM}, is, independently, hydrogen; cyclic or acyclic, branched or unbranched, substituted or unsubstituted aliphatic; cyclic or acyclic, branched or unbranched, substituted or unsubstituted heteroaliphatic; substituted or unsubstituted aryl; substituted or unsubstituted heteroaryl; substituted or unsubstituted acyl; substituted or unsubstituted hydroxyl; substituted or unsubstituted thiol; substituted or unsubstituted amino; azido; cyano; isocyano; halo; or nitro.

In certain embodiments, each instance of R^{KL}, R^{LL}, and R^{LM}, is, independently, hydrogen; cyclic or acyclic, branched or unbranched, substituted or unsubstituted aliphatic; cyclic or acyclic, branched or unbranched, substituted or unsubstituted heteroaliphatic; substituted or unsubstituted aryl; substituted or unsubstituted heteroaryl; substituted or unsubstituted acyl; substituted or unsubstituted hydroxyl; substituted or unsubstituted thiol; substituted or unsubstituted amino; cyano; isocyano: halo; or nitro.

In certain embodiments, p is 0. In certain embodiments, p is 1. In certain embodiments, p is 2. In certain embodiments, p is 3. In certain embodiments, p is 4. In certain embodiments, p is 5. In certain embodiments, p is 6. In certain embodiments, p is 7. In certain embodiments, p is 8. In certain embodiments, p is 9. In certain embodiments, p is 10.

In certain embodiments, each instance of y and z are, independently, 2, 3, 5, or 6.

In certain embodiments, both y and z are 2. In certain embodiments, both y and z are 3. In certain embodiments, both y and z are 5. In certain embodiments, both y and z are 6.

In certain embodiments, y is 2 and z is 3. In certain embodiments, y is 2 and z is 5. In certain embodiments, y is 2 and z is 6.

In certain embodiments, y is 3 and z is 2. In certain embodiments, y is 3 and z is 5. In certain embodiments, y is 3 and z is 6.

In certain embodiments, y is 5 and z is 2. In certain embodiments, y is 5 and z is 3. In certain embodiments, y is 5 and z is 6.

In certain embodiments, y is 6 and z is 2. In certain embodiments, y is 6 and z is 3. In certain embodiments, y is 6 and z is 5.

Exemplary amino acids of formula (AA) include, but are not limited to, those as depicted below, wherein R^{a}, R^{f}, and R^{E} are defined above and herein. In certain embodiments, R^{a} is hydrogen, and R^{f} is a suitable amino protecting group. In certain embodiments, R^{a} is hydrogen, and R^{f} is -Boc or -Fmoc. In certain embodiments, both R^{a} and R^{f} are suitable amino protecting groups. In certain embodiments, both R^{a} and R^{f} are hydrogen. In certain embodiments, R^{E} is hydrogen.

In certain embodiments, K is optionally substituted alkyl and L₁ is optionally substituted alkylene. In certain embodiments, K is unsubstituted C₁₋₆ alkyl and L₁ is optionally substituted C₁₋₁₀ alkylene. In certain embodiments, K is unsubstituted C₁₋₆ alkyl and L₁ is unsubstituted straight chain C₁₋₁₀ alkylene. In certain embodiments, L₁ is unsubstituted straight chain C₂₋₁₀ alkylene. In certain embodiments, L₁ is unsubstituted straight chain C₃₋₁₀ alkylene. In certain embodiments, L₁ is unsubstituted straight chain C₄₋₁₀ alkylene. In certain embodiments, L₁ is unsubstituted straight chain C₅₋₁₀ alkylene. In certain embodiments, L₁ is unsubstituted straight chain C₆₋₁₀alkylene. In certain embodiments, L₁ is unsubstituted straight chain C₇₋₁₀ alkylene. In certain embodiments, L₁ is unsubstituted straight chain C₈₋₁₀ alkylene. In certain embodiments, L₁ is unsubstituted straight chain C₉₋₁₀ alkylene.

In certain embodiments, additional modifications of the stabilized polypeptides include click chemistry reaction, reduction, oxidation, and nucleophilic or electrophilic additions to the double bond or triple bond provided from a metathesis reaction to provide a synthetically modified polypeptide. Other modifications may include conjugation of a stapled polypeptide, or a synthetically modifying the stapled polypeptide with a therapeutically active agent, label, or diagnostic agent anywhere on the stapled polypeptide scaffold, e.g., such as at the N-terminus of the stapled polypeptide, the C-terminus of the stapled polypeptide, on an amino acid side chain of the stapled polypeptide, or at one or more modified or unmodifed stapled sites (i.e., to a staple). Such modification may be useful in delivery of the peptide or therapeutically active agent to a cell, tissue, or organ. Such modifications may, in certain embodiments, allow for targeting to a particular type of cell or tissue.

In certain embodiments, the stabilized polypeptide described undergoes post-RCM modification or post-click chemistry modification. In certain embodiments, the alkynylene cross-linker undergoes post ring-closing metathesis (RCM) modifications such as click chemistry reaction (e.g. with an optionally substituted azide), reduction, or addition of a targeting moiety.

In certain embodiments, the alkynylene cross-linker undergoes post ring-closing metathesis (RCM) modifications such as click chemistry reaction (e.g. with an optionally substituted azide), reduction, or addition of a targeting moiety.

In certain embodiments, the stabilized polypeptide having an alkylene moiety further undergoes click chemistry reaction to react with an optionally azide of the formula R^{az}—N₃, wherein R^{az} is optionally substituted alkyl. In certain embodiments, R^{az} is optionally substituted C₁₋₈ alkyl. In certain embodiments, R^{az} is substituted C₁₋₈ alkyl. In certain embodiments, R^{az} is unsubstituted C₁₋₈ alkyl. In certain embodiments, R^{az} is unsubstituted, straight chain C₁₋₈ alkyl.

In certain embodiments, the reduction of the staple or stitch in the polypeptide can be carried out under a catalyst (e.g. Pd catalyst such as Pd₂(dba)₃) to provide an optionally substituted alkylene cross-linker. In certain embodiments, the reduction of the stitch in the polypeptide can be carried out under catalyst (e.g. Lindlar) to provide an optionally substituted alkenylene cross-linker. In certain embodiments, the optionally substituted alkenylene cross-linker is cis. In certain embodiments, the optionally substituted alkenylene cross-linker is trans.

In another aspect, provided herein is an amino acid having Formula (AA):

wherein:

R^{k} is cyclic or acyclic, branched or unbranched, substituted or unsubstituted alkyl; cyclic or acyclic, branched or unbranched, substituted or unsubstituted alkenyl; cyclic or acyclic, branched or unbranched, substituted or unsubstituted alkynyl; cyclic or acyclic, branched or unbranched, substituted or unsubstituted heteroalkyl; cyclic or acyclic, branched or unbranched, substituted or unsubstituted heteroalkenyl; cyclic or acyclic, branched or unbranched, substituted or unsubstituted heteroalkynyl; substituted or unsubstituted aryl; or substituted or unsubstituted heteroaryl;

L₁ is, independently, a bond, cyclic or acyclic, branched or unbranched, substituted or unsubstituted alkylene; cyclic or acyclic, branched or unbranched, substituted or unsubstituted alkenylene; cyclic or acyclic, branched or unbranched, substituted or unsubstituted alkynylene; cyclic or acyclic, branched or unbranched, substituted or unsubstituted heteroalkylene; cyclic or acyclic, branched or unbranched, substituted or unsubstituted heteroalkenylene; cyclic or acyclic, branched or unbranched, substituted or unsubstituted heteroalkynylene; substituted or unsubstituted arylene; substituted or unsubstituted heteroarylene; or substituted or unsubstituted acylene;
R^{a} is, independently, hydrogen; cyclic or acyclic, branched or unbranched, substituted or unsubstituted aliphatic; cyclic or acyclic, branched or unbranched, substituted or unsubstituted heteroaliphatic; substituted or unsubstituted aryl; substituted or unsubstituted heteroaryl; cyclic or acyclic, substituted or unsubstituted acyl; or R^{a} is a suitable amino protecting group;

R^{e}, is, independently, hydrogen; cyclic or acyclic, branched or unbranched, substituted or unsubstituted aliphatic; cyclic or acyclic, branched or unbranched, substituted or unsubstituted heteroaliphatic; substituted or unsubstituted aryl; substituted or unsubstituted heteroaryl; cyclic or acyclic, substituted or unsubstituted acyl; substituted or unsubstituted hydroxyl; substituted or unsubstituted thiol; substituted or unsubstituted amino; cyano; isocyano; halo; or nitro;

R^{E} is, independently, hydrogen, cyclic or acyclic, branched or unbranched, substituted or unsubstituted aliphatic; cyclic or acyclic, branched or unbranched, substituted or unsubstituted heteroaliphatic; substituted or unsubstituted aryl; substituted or unsubstituted heteroaryl; substituted or unsubstituted acyl; a resin; a suitable hydroxyl, amino, or thiol protecting group; or two R^{E} groups together form a substituted or unsubstituted 5- to 6-membered heterocyclic or heteroaromatic ring; and

R^{f} is, independently, hydrogen; cyclic or acyclic, branched or unbranched, substituted or unsubstituted aliphatic; cyclic or acyclic, branched or unbranched, substituted or unsubstituted heteroaliphatic; substituted or unsubstituted aryl; substituted or unsubstituted heteroaryl; substituted or unsubstituted acyl; a resin; a suitable amino protecting group; or R^{f} and R^{a} together form a substituted or unsubstituted 5- to 6-membered heterocyclic or heteroaromatic ring.

In certain embodiments, R^{k} is optionally substituted alkyl. In certain embodiments, R^{k} is unsubstituted alkyl (e.g. methyl or ethyl). In certain embodiments, R^{k} is unsubstituted C₁₋₁₀ alkyl. In certain embodiments, R^{k} is substituted C₁₋₁₀ alkyl.

In certain embodiments, R^{k} is optionally substituted alkyl; L₁ is cyclic or acyclic, branched or unbranched, substituted or unsubstituted alkylene; and R^{e} is cyclic or acyclic, branched or unbranched, substituted or unsubstituted aliphatic. In certain embodiments, R^{k} is unsubstituted alkyl; L₁ is straight chain, substituted or unsubstituted alkylene; and R^{e} is cyclic or acyclic, branched or unbranched, substituted or unsubstituted alkyl. In certain embodiments, R^{k} is unsubstituted alkyl; L₁ is straight chain unsubstituted alkylene; and R^{e} is straight chain unsubstituted alkyl (e.g. methyl or ethyl). In certain embodiments, L₁ is straight chain unsubstituted C₁₋₁₀ alkylene. In certain embodiments, L₁ is straight chain unsubstituted C₂₋₁₀ alkylene. In certain embodiments, L₁ is straight chain unsubstituted C₃₋₁₀ alkylene. In certain embodiments, L₁ is straight chain unsubstituted C₄₋₁₀ alkylene. In certain embodiments, L₁ is straight chain unsubstituted C₅₋₁₀ alkylene. In certain embodiments, L₁ is straight chain unsubstituted C₆₋₁₀ alkylene. In certain embodiments, L₁ is straight chain unsubstituted C₇₋₁₀ alkylene. In certain embodiments, L₁ is straight chain unsubstituted C₈₋₁₀ alkylene. In certain embodiments, L₁ is straight chain unsubstituted C₉₋₁₀ alkylene. In certain embodiments, L₁ is straight chain unsubstituted C₁₀ alkylene.

In another aspect, provided herein is a method of making a polypeptide having a stabilized alpha helix and a stabilized beta hairpin, said method comprising the steps of:
(i) providing a bis-amino acid of the formula (A):

(ii) providing an amino acid of the formula (B):

(iii) providing an amino acid of the formula (C):

(iv) providing an amino acid of the formula (D):

(v) providing an amino acid of the formula (E):

(vi) providing at least one additional amino acid;
(vii) coupling said amino acids of formulae (A), (B), (C), (D), and (E) with at least one amino acid of step (vi) to provide a precursor peptide.

In another aspect, provided herein is a method of making a polypeptide having a stabilized alpha helix and a stabilized beta hairpin, said method comprising the steps of:
(i) providing an amino acid of the formula (B):

(ii) providing an amino acid of the formula (C):

(iii) providing an amino acid of the formula (D):

(iv) providing an amino acid of the formula (E):

(v) providing at least one additional amino acid;
(vi) coupling said amino acids of formulae (B), (C), (D), and (E) with at least one amino acid of step (v) to provide a precursor peptide.

In certain embodiments, the method as described herein further comprising the steps of treating the precursor polypeptide with a catalyst. In certain embodiments, the method as described herein further comprising the steps of treating the precursor polypeptide with a RCM catalyst. In certain embodiments, the catalyst is a ruthenium catalyst.

In another aspect, provided herein is a method of making a polypeptide having a stabilized alpha helix and a stabilized beta hairpin, said method comprising the steps of:
(i) providing a bis-amino acid of the formula (A):

(ii) providing an amino acid of the formula (B):

(iii) providing an amino acid of the formula (C):

(iv) providing at least one additional amino acid;
(v) coupling said amino acids of formulae (A), (B), and (C) with at least one amino acid of step (iv) to provide a precursor peptide having an alpha helix;
(vi) providing an amino acid of the formula (D):

(vii) providing an amino acid of the formula (E):

(viii) providing at least one additional amino acid;
(ix) coupling said amino acids of formulae (D) and (E) with at least one amino acid of step (vi) to provide a precursor peptide having a beta hairpin;
(x) coupling the precursor peptide having an alpha helix with the precursor peptide having a beta hairpin to generate precursor peptide having an alpha helix and a beta hairpin.

In another aspect, provided herein is a method of making a polypeptide having a stabilized alpha helix and a stabilized beta hairpin, said method comprising the steps of:
(i) providing an amino acid of the formula (B):

(ii) providing an amino acid of the formula (C):

(iii) providing at least one additional amino acid;
(iv) coupling said amino acids of formulae (B) and (C) with at least one amino acid of step (iii) to provide a precursor peptide having an alpha helix;
(v) providing an amino acid of the formula (D):

(vi) providing an amino acid of the formula (E):

(vii) providing at least one additional amino acid;
(viii) coupling said amino acids of formulae (D) and (E) with at least one amino acid of step (vii) to provide a precursor peptide having a beta hairpin;
(ix) coupling the precursor peptide having an alpha helix with the precursor peptide having a beta hairpin to generate precursor peptide having an alpha helix and a beta hairpin.

In certain embodiments, the method further comprises the steps of treating the precursor polypeptide having an alpha helix and a beta hairpin with a RCM catalyst as described herein. In certain embodiments, the method further comprises steps of treating the precursor polypeptide or precursor peptide having an alpha helix and a beta hairpin with a click chemistry reagent. In certain embodiments, the click chemistry reagent is a copper reagent. In certain embodiments, treatment with a click chemistry reagent is after the treatment with the RCM catalyst. In certain embodiments, treatment with a click chemistry reagent is before the treatment with the RCM catalyst.

In certain embodiments, the method comprises a solution phase synthesis of an inventive polypeptide. Solution phase synthesis, as mentioned above, is a well-known technique for the construction of polypeptides. An exemplary solution phase synthesis comprises the steps of: (1) providing an amino acid protected at the N-terminus with a suitable amino protecting group: (2) providing an amino acid protected at the C-terminus with a suitable carboxylic acid protecting group; (3) coupling the N-protected amino acid to the C-protected amino acid; (4) deprotecting the product of the coupling reaction; and (5) repeating steps (3) to (4) until a desired polypeptide is obtained, wherein at least two of the amino acids coupled at any of the above steps each comprise at least one terminally unsaturated amino acid sidechain, and at least one α,α-disubstituted amino acid comprises two terminally unsaturated amino acid side chains. During the course of the above synthesis, various parameters can be varied, including, but not limited to placement of amino acids with terminally unsaturated side chains, stereochemistry of amino acids, terminally unsaturated side chain length and functionality, and amino acid residues utilized.

In certain embodiments, the method comprises a solid phase synthesis of an inventive polypeptide. Solid phase synthesis, as mentioned above, is a well-known technique for the construction of polypeptides. An exemplary solid phase synthesis comprises the steps of: (1) providing a resin-bound amino acid; (2) deprotecting the resin bound amino acid; (3) coupling an amino acid to the deprotected resin-bound amino acid; (4) repeating steps (3) until a desired peptide is obtained, wherein at least two of the amino acids coupled at any of the above steps each comprise at least one terminally unsaturated amino acid sidechain, and at least one α,α-disubstituted amino acid comprises two terminally unsaturated amino acid side chains. During the course of the above synthesis, various parameters can be varied, including, but not limited to placement of amino acids with terminally unsaturated side chains, stereochemistry of amino acids, terminally unsaturated side chain length and functionality, and amino acid residues utilized.

After a desired polypeptide is synthesized using an appropriate technique, the polypeptide is contacted with a specific catalyst to promote “stitching” of the polypeptide. For example, the resin-bound polypeptide may be contacted with a catalyst to promote “stitching,” or may first be cleaved from the resin, and then contacted with a catalyst to promote “stitching.”

Different amino acids have different propensities for forming different secondary structures. For example, methionine (M), alanine (A), leucine (L), glutamate (E), and lysine (K) all have especially high alpha-helix forming propensities. In contrast, proline (P) and glycine (G) are alpha-helix disrupters. Thus, in certain embodiments, the at least one amino acid of step (iv) refers to a group selected from alanine, arginine, asparagine, aspartic acid, cysteine, glutamic acid, glutamine, histidine, isoleucine, leucine, lysine, methionine, phenylalanine, serine, threonine, tryptophan, tyrosine, and valine.

In certain embodiments, the coupling step comprises the use of a coupling reagent.

Exemplary coupling reagents include, but are not limited to, benzotriazol-1-yloxy-tris(dimethylamino)-phosphonium hexafluorophosphate (BOP), benzotriazole-1-yl-oxy-tris-pyrrolidino-phosphonium hexafluorophosphate (PyBOP), bromo-tris-pyrrolidino phosphonium hexafluorophosphate (PyBroP), 1-ethyl-3-(3-dimethyllaminopropyl) carbodiimide (EDC), N,N′-carbonyldiimidazole (CDI), 3-(diethoxyphosphoryloxy)-1,2,3-benzotriazin-4(3H)-one (DEPBT), 1-hydroxy-7-azabenzotriazole (HOAt), 1-hydroxy-7-benzotriazole (HOBt), 2-(7-aza-1H-benzotriazole-1-yl)-1,1,3,3-tetramethyluronium hexafluorophosphate (HATU), 2-(6-chloro-1H-benzotriazole-1-yl)-1,1,3,3-tetramethylaminium hexafluorophosphate (HCTU), 2-(1H-benzotriazole-1-yl)-1,1,3,3-tetramethyluronium hexafluorophosphate (HBTU), O-(7-azabenzotriazole-1-yl)-N,N,N′,N′-tetramethyluronium tetrafluoroborate (TATU), 2-(1H-benzotriazole-1-yl)-1,1,3,3-tetramethyluronium tetrafluoroborate (TBTU), N,N,N′,N′-tetramethyl-O-(3,4-dihydro-4-oxo-1,2,3-benzotriazin-3-yl)uranium tetrafluoroborate (TDBTU), and O—(N-succinimidyl)-1,1,3,3-tetramethyl uranium tetrafluoroborate (TSTU)).

In certain embodiments, the coupling step further comprises a suitable base. Suitable bases include, but are not limited to, potassium carbonate, potassium hydroxide, sodium hydroxide, tetrabutylammonium hydroxide, benzyltrimethylammonium hydroxide, triethylbenzylammonium hydroxide, 1,1,3,3-tetramethylguanidine, 1,8-diazabicyclo[5.4.0]undec-7-ene (DBU), N-methylmorpholine, diisopropylethylamine (DIPEA), tetramethylethylenediamine (TMEDA), pyridine (Py), 1,4-diazabicyclo[2.2.2]octane (DABCO), N,N-dimethylamino pyridine (DMAP), or triethylamine (NEt₃).

In certain embodiments, the coupling step is carried out in a suitable medium. A suitable medium is a solvent or a solvent mixture that, in combination with the combined reacting partners and reagents, facilitates the progress of the reaction therebetween. A suitable solvent may solubilize one or more of the reaction components, or, alternatively, the suitable solvent may facilitate the suspension of one or more of the reaction components; see generally, March's Advanced Organic Chemistry: Reactions, Mechanisms, and Structure, M. B. Smith and J. March, 5 Edition, John Wiley & Sons, 2001, and Comprehensive Organic Transformations, R. C. Larock, 2^{nd} Edition, John Wiley & Sons, 1999, the entire contents of each of which are incorporated herein by reference. Suitable solvents for include ethers, halogenated hydrocarbons, aromatic solvents, polar aprotic solvents, or mixtures thereof. In other embodiments, the solvent is diethyl ether, dioxane, tetrahydrofuran (THF), dichloromethane (DCM), dichloroethane (DCE), acetonitrile (ACN), chloroform, toluene, benzene, dimethylformamide (DMF), dimethylacetamide (DMA), dimethylsulfoxide (DMSO), N-methyl pyrrolidinone (NMP), or mixtures thereof.

In other embodiments, the coupling step is conducted at suitable temperature, such as between about 0° C. and about 100° C.

In certain embodiments, the RCM catalyst is a tungsten (W), molybdenum (Mo), or ruthenium (Ru) catalyst. In certain embodiments, the RCM catalyst is a ruthenuim catalyst. Suitable RCM catalysts employable by the above synthetic method include catalysts are as depicted below, and as described in see Grubbs et al., Acc. Chem. Res. 1995, 28, 446-452; U.S. Pat. No. 5,811,515; Schrock et al., Organometallics (1982) 1 1645; Gallivan et al., Tetrahedron Letters (2005) 46:2577-2580; Furstner et al., J. Am. Chem. Soc. (1999) 121:9453; and Chem. Eur. J. (2001) 7:5299; the entire contents of each of which are incorporated herein by reference.

In certain embodiments, the RCM catalyst is a Schrock catalyst. In certain embodiments, the Schrock catalyst is selected from any of the following:

In certain embodiments, the RCM catalyst is a Grubbs catalyst. In certain embodiments, the Grubbs catalyst is selected from any of the following:

X=Cl; Br; I

Cy=cyclohexyl
Benzylidenebis-(tricyclohexylphosphine)-dichlororuthenium (X=Cl)
Benzylidenebis-(tricyclohexylphosphine)-dibromoruthenium (X=Br)
Benzlidenebis-(tricyclohexylphosphine)-diiodoruthenium (X=I);

X=Cl; Br, I
R=cyclohexyl (Cy); phenyl (Ph); benzyl (Bn)
1,3-(Bis(mesityl)-2-imidazolidinylidene)dichloro-(phenylmethylene) (tricyclohexyl-phosphine)ruthenium (X=Cl; R=cyclohexyl)
1,3-(Bis(mesityl)-2-imidazolidinylidene)dibromo-(phenylmethylene) (tricyclohexyl-phosphine)ruthenium (X=Br; R=cyclohexyl)
1,3-(Bis(mesityl)-2-imidazolidinylidene)diiodo-(phenylmethylene) (tricyclohexyl-phosphine)ruthenium (X=I; R=cyclohexyl)
1,3-(Bis(mesityl)-2-imidazolidinylidene)dichloro-(phenylmethylene) (triphenylphosphine)ruthenium (X=Cl; R=phenyl)
1,3-(Bis(mesityl)-2-imidazolidinylidene)dichloro-(phenylmethylene) (tribenzylphosphine)ruthenium (X=Cl; R=benzyl);

In certain embodiments, the RCM catalyst is a Grubbs-Hoveyda catalyst. In certain embodiments, the Grubbs-Hoveyda catalyst is selected from any of the following:

In certain embodiments, the RCM catalyst is selected from any of the following:

and

In certain embodiments, the RCM catalyst is a tungsten catalyst (e.g. Tris(t-butoxy) tungsten neopentylidyne). In certain embodiments, the RCM catalyst is a molybdenum catalyst (e.g. Tris(triphenylsilyloxy) molybdenum nitride pyridine complex) (J. Am. Chem. Soc., 2010, 132, 11045-11057; J. Am. Chem. Soc., 2009, 131, 9468).

It will also be appreciated, that in addition to RCM catalysts, other reagents capable of promoting carbon-carbon bond formation can also be utilized. For example, other reactions that can be utilized, include, but are not limited to palladium coupling reactions, transition metal catalyzed cross coupling reactions, pinacol couplings (terminal aldehydes), hydrozirconation (terminal alkynes), nucleophilic addition reactions, and NHK (Nozaki-Hiyama-Kishi (Furstner et al., J. Am. Chem. Soc. 1996, 118, 12349)) coupling reactions. Thus, the appropriate reactive moieties are first incorporated into desired amino acids or unnatural amino acids, and then the peptide is subjected to reaction conditions to effect “stitching” and subsequent stabilization of a desired secondary structure.

The present invention provides pharmaceutical compositions comprising a polypeptide as described herein, and optionally a pharmaceutically acceptable carrier. Pharmaceutical compositions comprise compositions for therapeutic use as well as cosmetic compositions. Such compositions may optionally comprise one or more additional therapeutically active agents. In accordance with some embodiments, a method of administering a pharmaceutical composition comprising an inventive pharmaceutical composition to a subject in need thereof is provided. In some embodiments, the inventive composition is administered to humans.

In one aspect, the present invention provides a method of treating a disorder in a subject in need thereof, comprising administering an effective amount of a provided polypeptide, or pharmaceutical composition thereof, to the subject.

In another aspect, the present invention provides a method of modulating STAT signaling pathway in a biological sample comprising administering, contacting, or applying an effective amount of a provided polypeptide, or pharmaceutical composition thereof, to the biological sample.

In another aspect, the present invention provides a method of inducing apoptosis of a cell in a biological sample, the method comprising administering, contacting, or applying an effective amount of a provided polypeptide, or pharmaceutical composition thereof, to the biological sample.

Exemplary disorders include, but are not limited to, proliferative disorders, neurological disorders, immunological disorders, endocrinologic disorders, cardiovascular disorders, hematologic disorders, inflammatory disorders, and disorders characterized by premature or unwanted cell death.

As used herein, a proliferative disorder includes, but is not limited to, cancer, hematopoietic neoplastic disorders, proliferative breast disease, proliferative disorders of the lung, proliferative disorders of the colon, proliferative disorders of the liver, and proliferative disorders of the ovary.

Exemplary cancers include, but are not limited to, carcinoma, sarcoma, or metastatic disorders, breast cancer, ovarian cancer, colon cancer, lung cancer, fibrosarcoma, myosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, gastric cancer, esophageal cancer, rectal cancer, pancreatic cancer, ovarian cancer, prostate cancer, uterine cancer, cancer of the head and neck, skin cancer, brain cancer, squamous cell carcinoma, sebaceous gland carcinoma, papillary carcinoma, papillary adenocarcinoma, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular cancer, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemia, lymphoma, and Kaposi's sarcoma.

Exemplary hematopoietic neoplastic disorders include, but are not limited to, disorders involving hyperplastic/neoplastic cells of hematopoietic origin, e.g., arising from myeloid, lymphoid or erythroid lineages, or precursor cells thereof. In certain embodiments, the disorders arise from poorly differentiated acute leukemias, e.g., erythroblastic leukemia and acute megakaryoblastic leukemia. Additional exemplary myeloid disorders include, but are not limited to, acute promyeloid leukemia (APML), acute myelogenous leukemia (AML) and chronic myelogenous leukemia (CML); lymphoid malignancies include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to non-Hodgkin lymphoma and variants thereof, peripheral T-cell lymphomas, adult T cell leukemia/lymphoma (ATL), cutaneous T-cell lymphoma (CTCL), large granular lymphocytic leukemia (LGF), Hodgkin's disease, and Reed-Stemberg disease.

Exemplary proliferative breast diseases include, but are not limited to, epithelial hyperplasia, sclerosing adenosis, and small duct papillomas; tumors, e.g., stromal tumors such as fibroadenoma, phyllodes tumor, and sarcomas, and epithelial tumors such as large duct papilloma; carcinoma of the breast including in situ (noninvasive) carcinoma that includes ductal carcinoma in situ (including Paget's disease) and lobular carcinoma in situ, and invasive (infiltrating) carcinoma including, but not limited to, invasive ductal carcinoma, invasive lobular carcinoma, medullary carcinoma, colloid (mucinous) carcinoma, tubular carcinoma, and invasive papillary carcinoma, and miscellaneous malignant neoplasms. Disorders in the male breast include, but are not limited to, gynecomastia and carcinoma.

Exemplary proliferative disorders of the lung include, but are not limited to, bronchogenic carcinoma, including paraneoplastic syndromes, bronchioloalveolar carcinoma, neuroendocrine tumors, such as bronchial carcinoid, miscellaneous tumors, and metastatic tumors; pathologies of the pleura, including inflammatory pleural effusions, noninflammatory pleural effusions, pneumothorax, and pleural tumors, including solitary fibrous tumors (pleural fibroma) and malignant mesothelioma.

Exemplary proliferative disorders of the colon include, but are not limited to, non-neoplastic polyps, adenomas, familial syndromes, colorectal carcinogenesis, colorectal carcinoma, and carcinoid tumors.

Exemplary proliferative disorders of the liver include, but are not limited to, nodular hyperplasias, adenomas, and malignant tumors, including primary carcinoma of the liver and metastatic tumors.

Exemplary proliferative disorders of the ovary include, but are not limited to, ovarian tumors such as, tumors of coelomic epithelium, serous tumors, mucinous tumors, endometeriod tumors, clear cell adenocarcinoma, cystadenofibroma, brenner tumor, surface epithelial tumors; germ cell tumors such as mature (benign) teratomas, monodermal teratomas, immature malignant teratomas, dysgerminoma, endodermal sinus tumor, choriocarcinoma; sex cord-stomal tumors such as, granulosa-theca cell tumors, thecomafibromas, androblastomas, hill cell tumors, and gonadoblastoma; and metastatic tumors such as Krukenberg tumors.

Additional description of the related peptide stapling or peptide stitching can be found in WO2011/008260, WO2010/011313, WO2008/121767, WO2012/040459, WO2012/174423, and PCT/US2013/062004, U.S. Ser. No. 61/478,845, 61/478862, 61/705950, 61/789,157, and 61/708,371, all of which are incorporated by reference herein.

This application refers to various issued patents, published patent applications, journal articles, and other publications, all of which are incorporated herein by reference.

### EXAMPLES

### Synthesis and Stabilization of the α-Helical Part of the Combined Motif

As a first step towards the synthesis of the stabilized miniature proteins, the stabilizing properties of all-hydrocarbon bridges on the conformation of the α-helical part of the motif were examined. Therefore a panel of all-hydrocarbon stapled STAT3 SH2 peptides (SABS) featuring i,i+4 as well as i,i+7 staples at different positions of the α-helical portion were synthesized (FIG. 6, αA helix). Peptides containing an i,i+7 staple exhibited cis/trans stereoisomers in respect to the double bonds of the all-hydrocarbon bridge and were separated for evaluation (e.g. named SABS_{E1} and SABS_{E2}). To identify the optimal staple compositions, we scanned the conformational and cell-penetrating properties of the different candidates.

Conformational Analysis.

The conformation of the synthesized peptides was examined using circular dichroism (CD) spectroscopy. Spectra were recorded in phosphate buffer (PBS) at pH 7.4, reflecting the physiological conditions for following cell experiments. The measurements were done at 20° C. with N-acetylated, C-amidated peptides at concentrations of 100 μM, showing good solubility of the modified peptides in aqueous media. Our data shows that all but one peptide (SABS_{F1}) display characteristics of an α-helical structure (minima at 208 and 222 nm) in comparison to the wildtype STAT3 SH2 peptide (FIG. 7).

Cell-Penetrating Properties.

Initial cell-penetration assays of selected fluorescently labeled stapled α-helical peptides were performed via flow cytometry with Jurkat cells at a peptide concentration of 5 μM at 37° C. for 3 h of incubation time. FIG. 8 indicates an increased cellular uptake of the i,i+4 stapled peptide SABS_{A} and the i,i+7 stapled peptide SABS-F2 (cis/trans isomer SABS_{F2} compared to wildtype STAT3 SH2.

The experimental data of the stabilized α-helical peptides (SABS_{A}-SABS_{G}) of the STAT3 SH2 motif generated thus far indicate a higher conformational stability as well as cell penetrating properties compared to the unmodified wildtype peptide STAT3 SH2 (FIGS. 7 and 8).

1. A polypeptide comprising a stabilized alpha helix and at least one other stabilized structural motif that is not an alpha helix.

2. The polypeptide of claim 1, wherein the polypeptide comprises a stabilized alpha helix and a stabilized beta hairpin.

3-13. (canceled)

14. The polypeptide of claim 1, wherein the polypeptide is a STAT peptide or a derivative thereof.

15. (canceled)

16. The polypeptide of claim 1, wherein the polypeptide comprises a STAT3 SH2 peptide (ISKERERAILSTKPPGTFLLRFSESSKEGGVTFTWV), or a derivative thereof.

17. (canceled)

18. The polypeptide of claim 1, wherein the polypeptide is one of the following peptides:

19. The polypeptide of claim 1, wherein the polypeptide is one of the following peptides:

20-21. (canceled)

22. A polypeptide comprising a stabilized alpha helix, wherein the polypeptide is a STAT peptide or a derivative thereof.

23. The polypeptide of claim 22, wherein the polypeptide is a STAT3 peptide or a derivative thereof.

24. The polypeptide of claim 22, wherein the polypeptide comprises a STAT3 SH2 peptide (ISKERERAILSTKPPGTFLLRFSESSKEGGVTFTWV) or a derivative thereof.

25. The polypeptide of claim 24, wherein the STAT3 SH2 peptide derivative is derived from
ISKERERAILSTKPPGTFLLRFSESSpPGGVTFTWV or
ISKERERAILSTKPPGTFLLRFSESPpEGGVTFTWV.

26. The polypeptide of claim 22, wherein the polypeptide is one of the following peptides:

27. The polypeptide of claim 1, wherein the polypeptide is one of the following peptides:

28. The polypeptide of claim 1, wherein the polypeptide is one of the following peptides:

29. (canceled)

30. A polypeptide of one of the following formulae:
wherein:
each instance of K, L₁, L₂, and M, is, independently, a bond, cyclic or acyclic, branched or unbranched, substituted or unsubstituted alkylene; cyclic or acyclic, branched or unbranched, substituted or unsubstituted alkenylene; cyclic or acyclic, branched or unbranched, substituted or unsubstituted alkynylene; cyclic or acyclic, branched or unbranched, substituted or unsubstituted heteroalkylene; cyclic or acyclic, branched or unbranched, substituted or unsubstituted heteroalkenylene; cyclic or acyclic, branched or unbranched, substituted or unsubstituted heteroalkynylene; substituted or unsubstituted arylene; substituted or unsubstituted heteroarylene; or substituted or unsubstituted acylene;
each instance of R^{a} is, independently, hydrogen; cyclic or acyclic, branched or unbranched, substituted or unsubstituted aliphatic; cyclic or acyclic, branched or unbranched, substituted or unsubstituted heteroaliphatic; substituted or unsubstituted aryl; substituted or unsubstituted heteroaryl; cyclic or acyclic, substituted or unsubstituted acyl; or R^{a} is a suitable amino protecting group;
each instance of R^{b} is, independently, a suitable amino acid side chain; hydrogen; cyclic or acyclic, branched or unbranched, substituted or unsubstituted aliphatic; cyclic or acyclic, branched or unbranched, substituted or unsubstituted heteroaliphatic; substituted or unsubstituted aryl; substituted or unsubstituted heteroaryl; cyclic or acyclic, substituted or unsubstituted acyl; substituted or unsubstituted hydroxyl; substituted or unsubstituted thiol; substituted or unsubstituted amino; cyano; isocyano; halo; or nitro;
each instance of R^{c}, is, independently, hydrogen; cyclic or acyclic, branched or unbranched, substituted or unsubstituted aliphatic; cyclic or acyclic, branched or unbranched, substituted or unsubstituted heteroaliphatic; substituted or unsubstituted aryl; substituted or unsubstituted heteroaryl; cyclic or acyclic, substituted or unsubstituted acyl; substituted or unsubstituted hydroxyl; substituted or unsubstituted thiol; substituted or unsubstituted amino; cyano; isocyano; halo; or nitro;
each instance of R^{e} is, independently, —R^{E}, —OR^{E}, —N(R^{E})₂, or —SR^{E}, wherein each instance of R^{E} is, independently, hydrogen, cyclic or acyclic, branched or unbranched, substituted or unsubstituted aliphatic; cyclic or acyclic, branched or unbranched, substituted or unsubstituted heteroaliphatic; substituted or unsubstituted aryl; substituted or unsubstituted heteroaryl; substituted or unsubstituted acyl; a resin; a suitable hydroxyl, amino, or thiol protecting group; or two R^{E} groups together form a substituted or unsubstituted 5- to 6-membered heterocyclic or heteroaromatic ring;
each instance of R^{f} is, independently, hydrogen; cyclic or acyclic, branched or unbranched, substituted or unsubstituted aliphatic; cyclic or acyclic, branched or unbranched, substituted or unsubstituted heteroaliphatic; substituted or unsubstituted aryl; substituted or unsubstituted heteroaryl; substituted or unsubstituted acyl; a resin; a suitable amino protecting group; a label optionally joined by a linker, wherein the linker is selected from cyclic or acyclic, branched or unbranched, substituted or unsubstituted alkylene; cyclic or acyclic, branched or unbranched, substituted or unsubstituted alkenylene; cyclic or acyclic, branched or unbranched, substituted or unsubstituted alkynylene; cyclic or acyclic, branched or unbranched, substituted or unsubstituted heteroalkylene; cyclic or acyclic, branched or unbranched, substituted or unsubstituted heteroalkenylene; cyclic or acyclic, branched or unbranched, substituted or unsubstituted heteroalkynylene; substituted or unsubstituted arylene; substituted or unsubstituted heteroarylene; or substituted or unsubstituted acylene; or R^{f} and R^{a} together form a substituted or unsubstituted 5- to 6-membered heterocyclic or heteroaromatic ring;
R^{KL} is hydrogen; cyclic or acyclic, branched or unbranched, substituted or unsubstituted aliphatic; cyclic or acyclic, branched or unbranched, substituted or unsubstituted heteroaliphatic; substituted or unsubstituted aryl; substituted or unsubstituted heteroaryl; substituted or unsubstituted acyl; substituted or unsubstituted hydroxyl; substituted or unsubstituted thiol; substituted or unsubstituted amino; azido; cyano; isocyano; halo; nitro;
or two adjacent R^{KL} groups are joined to form a substituted or unsubstituted 5- to 8-membered cycloaliphatic ring; substituted or unsubstituted 5- to 8-membered cycloheteroaliphatic ring; substituted or unsubstituted aryl ring; or substituted or unsubstituted heteroaryl ring; two adjacent R^{KL} groups are joined to form a substituted or unsubstituted 5- to 8-membered cycloaliphatic ring; substituted or unsubstituted 5- to 8-membered cycloheteroaliphatic ring; substituted or unsubstituted aryl ring; or substituted or unsubstituted heteroaryl ring; or two adjacent R^{LM} groups are joined to form a substituted or unsubstituted 5- to 8-membered cycloaliphatic ring; substituted or unsubstituted 5- to 8-membered cycloheteroaliphatic ring; substituted or unsubstituted aryl ring; or substituted or unsubstituted heteroaryl ring;
A is —NH—, —NH—NH—, —NH—O—, —O—NH—, —S—, —O—,
Q is —NH—, —NH—NH—, —O—NH—, —NH—O—, —S—, or —O—;
W is O, S, or NR^{W1};
R^{W1} is hydrogen, optionally substituted alkyl; optionally substituted alkenyl; optionally substituted alkynyl; optionally substituted carbocyclyl; optionally substituted heterocyclyl; optionally substituted aryl; optionally substituted heteroaryl; or a nitrogen protecting group; and
R^{W2} is hydrogen, optionally substituted alkyl; optionally substituted alkenyl; optionally substituted alkynyl; optionally substituted carbocyclyl; optionally substituted heterocyclyl; optionally substituted aryl; optionally substituted heteroaryl, or two R^{W2} groups are joined to form an optionally substituted cyclic moiety;
each instance of X_{AA} is, independently, a natural or unnatural amino acid;
each instance of x is, independently, an integer between 0 to 3;
each instance of y is, independently, an integer between 2 to 8;
each instance of z1 and z2 is, independently, an integer between 2 to 30;
each instance of j is, independently, an integer between 1 to 10;
each instance of s and t is, independently, an integer between 0 and 100;
each instance of v is, independently, an integer between 0 to 4; and
corresponds to a single, double, or triple bond.

31. (canceled)

32. A polypeptide of Formula (III):
wherein:
each instance of K, L₁, L₂, and M, is, independently, a bond, cyclic or acyclic, branched or unbranched, substituted or unsubstituted alkylene; cyclic or acyclic, branched or unbranched, substituted or unsubstituted alkenylene; cyclic or acyclic, branched or unbranched, substituted or unsubstituted alkynylene; cyclic or acyclic, branched or unbranched, substituted or unsubstituted heteroalkylene; cyclic or acyclic, branched or unbranched, substituted or unsubstituted heteroalkenylene; cyclic or acyclic, branched or unbranched, substituted or unsubstituted heteroalkynylene; substituted or unsubstituted arylene; substituted or unsubstituted heteroarylene; or substituted or unsubstituted acylene;
each instance of R^{a} is, independently, hydrogen; cyclic or acyclic, branched or unbranched, substituted or unsubstituted aliphatic; cyclic or acyclic, branched or unbranched, substituted or unsubstituted heteroaliphatic; substituted or unsubstituted aryl; substituted or unsubstituted heteroaryl; cyclic or acyclic, substituted or unsubstituted acyl; or R^{a} is a suitable amino protecting group;
each instance of R^{b} is, independently, a suitable amino acid side chain; hydrogen; cyclic or acyclic, branched or unbranched, substituted or unsubstituted aliphatic; cyclic or acyclic, branched or unbranched, substituted or unsubstituted heteroaliphatic; substituted or unsubstituted aryl; substituted or unsubstituted heteroaryl; cyclic or acyclic, substituted or unsubstituted acyl; substituted or unsubstituted hydroxyl; substituted or unsubstituted thiol; substituted or unsubstituted amino; cyano; isocyano; halo; or nitro;
each instance of R^{e} is, independently, —R^{E}, —OR^{E}, —N(R^{E})₂, or —SR^{E}, wherein each instance of R^{E} is, independently, hydrogen, cyclic or acyclic, branched or unbranched, substituted or unsubstituted aliphatic; cyclic or acyclic, branched or unbranched, substituted or unsubstituted heteroaliphatic; substituted or unsubstituted aryl; substituted or unsubstituted heteroaryl; substituted or unsubstituted acyl; a resin; a suitable hydroxyl, amino, or thiol protecting group; or two R^{E} groups together form a substituted or unsubstituted 5- to 6-membered heterocyclic or heteroaromatic ring;
each instance of R^{f} is, independently, hydrogen; cyclic or acyclic, branched or unbranched, substituted or unsubstituted aliphatic; cyclic or acyclic, branched or unbranched, substituted or unsubstituted heteroaliphatic; substituted or unsubstituted aryl; substituted or unsubstituted heteroaryl; substituted or unsubstituted acyl; a resin; a suitable amino protecting group; a label optionally joined by a linker, wherein the linker is selected from cyclic or acyclic, branched or unbranched, substituted or unsubstituted alkylene; cyclic or acyclic, branched or unbranched, substituted or unsubstituted alkenylene; cyclic or acyclic, branched or unbranched, substituted or unsubstituted alkynylene; cyclic or acyclic, branched or unbranched, substituted or unsubstituted heteroalkylene; cyclic or acyclic, branched or unbranched, substituted or unsubstituted heteroalkenylene; cyclic or acyclic, branched or unbranched, substituted or unsubstituted heteroalkynylene; substituted or unsubstituted arylene; substituted or unsubstituted heteroarylene; or substituted or unsubstituted acylene; or R^{f} and R^{a} together form a substituted or unsubstituted 5- to 6-membered heterocyclic or heteroaromatic ring;
each instance of R^{KL}, R^{LL}, and R^{LM}, is, independently, hydrogen; cyclic or acyclic, branched or unbranched, substituted or unsubstituted aliphatic; cyclic or acyclic, branched or unbranched, substituted or unsubstituted heteroaliphatic; substituted or unsubstituted aryl; substituted or unsubstituted heteroaryl; substituted or unsubstituted acyl; substituted or unsubstituted hydroxyl; substituted or unsubstituted thiol; substituted or unsubstituted amino; azido; cyano; isocyano; halo; nitro;
or two adjacent R^{KL} groups are joined to form a substituted or unsubstituted 5- to 8-membered cycloaliphatic ring; substituted or unsubstituted 5- to 8-membered cycloheteroaliphatic ring; substituted or unsubstituted aryl ring; or substituted or unsubstituted heteroaryl ring; two adjacent R^{KL} groups are joined to form a substituted or unsubstituted 5- to 8-membered cycloaliphatic ring; substituted or unsubstituted 5- to 8-membered cycloheteroaliphatic ring; substituted or unsubstituted aryl ring; or substituted or unsubstituted heteroaryl ring; or two adjacent R^{LM} groups are joined to form a substituted or unsubstituted 5- to 8-membered cycloaliphatic ring; substituted or unsubstituted 5- to 8-membered cycloheteroaliphatic ring; substituted or unsubstituted aryl ring; or substituted or unsubstituted heteroaryl ring;
A is —NH—, —NH—NH—, —NH—O—, —O—NH—, —S—, —O—,
Q is —NH—, —NH—NH—, —O—NH—, —NH—O—, —S—, or —O—;
W is O, S, or NR^{W1};
R^{W1} is hydrogen, optionally substituted alkyl; optionally substituted alkenyl; optionally substituted alkynyl; optionally substituted carbocyclyl; optionally substituted heterocyclyl; optionally substituted aryl; optionally substituted heteroaryl; or a nitrogen protecting group; and
R^{W2} is hydrogen, optionally substituted alkyl; optionally substituted alkenyl; optionally substituted alkynyl; optionally substituted carbocyclyl; optionally substituted heterocyclyl; optionally substituted aryl; optionally substituted heteroaryl, or two R^{W2} groups are joined to form an optionally substituted cyclic moiety;
each instance of X_{AA} is, independently, a natural or unnatural amino acid;
each instance of x is, independently, an integer between 0 to 3;
each instance of y and z is, independently, an integer between 2 to 8;
each instance of z1 and z2 is, independently, an integer between 2 to 30;
each instance of j is, independently, an integer between 1 to 10;
each instance of p is, independently, an integer between 0 to 10;
each instance of s and t is, independently, an integer between 0 and 100;
each instance of u, v, and q, is, independently, an integer between 0 to 4;
and wherein:
corresponds to a single, double, or triple bond.

33-52. (canceled)

53. A pharmaceutical composition comprising a polypeptide of claim 1 and a pharmaceutically acceptable carrier.

54. (canceled)

55. A method of treating a disease, disorder, or condition in a subject, said method comprising administering a therapeutically effective amount of a polypeptide of claim 1 to a subject in need thereof.

56-58. (canceled)

59. A method of modulating STAT signaling pathway in a biological sample or subject comprising administering an effective amount of a polypeptide of claim 1 to the biological sample or subject.

60. A method of inducing apoptosis of a cell in a biological sample or subject, the method comprising administering an effective amount of a polypeptide of claim 1 to the biological sample or subject.

61. (canceled)

62. An amino acid having Formula (AA):
wherein:
R^{k} is cyclic or acyclic, branched or unbranched, substituted or unsubstituted alkyl; cyclic or acyclic, branched or unbranched, substituted or unsubstituted alkenyl; cyclic or acyclic, branched or unbranched, substituted or unsubstituted alkynyl; cyclic or acyclic, branched or unbranched, substituted or unsubstituted heteroalkyl; cyclic or acyclic, branched or unbranched, substituted or unsubstituted heteroalkenyl; cyclic or acyclic, branched or unbranched, substituted or unsubstituted heteroalkynyl; substituted or unsubstituted aryl; or substituted or unsubstituted heteroaryl;
L₁ is, independently, a bond, cyclic or acyclic, branched or unbranched, substituted or unsubstituted alkylene; cyclic or acyclic, branched or unbranched, substituted or unsubstituted alkenylene; cyclic or acyclic, branched or unbranched, substituted or unsubstituted alkynylene; cyclic or acyclic, branched or unbranched, substituted or unsubstituted heteroalkylene; cyclic or acyclic, branched or unbranched, substituted or unsubstituted heteroalkenylene; cyclic or acyclic, branched or unbranched, substituted or unsubstituted heteroalkynylene; substituted or unsubstituted arylene; substituted or unsubstituted heteroarylene; or substituted or unsubstituted acylene;
R^{a} is, independently, hydrogen; cyclic or acyclic, branched or unbranched, substituted or unsubstituted aliphatic; cyclic or acyclic, branched or unbranched, substituted or unsubstituted heteroaliphatic; substituted or unsubstituted aryl; substituted or unsubstituted heteroaryl; cyclic or acyclic, substituted or unsubstituted acyl; or R^{a} is a suitable amino protecting group;
R^{c}, is, independently, hydrogen; cyclic or acyclic, branched or unbranched, substituted or unsubstituted aliphatic; cyclic or acyclic, branched or unbranched, substituted or unsubstituted heteroaliphatic; substituted or unsubstituted aryl; substituted or unsubstituted heteroaryl; cyclic or acyclic, substituted or unsubstituted acyl; substituted or unsubstituted hydroxyl; substituted or unsubstituted thiol; substituted or unsubstituted amino; cyano; isocyano; halo; or nitro;
R^{E} is, independently, hydrogen, cyclic or acyclic, branched or unbranched, substituted or unsubstituted aliphatic; cyclic or acyclic, branched or unbranched, substituted or unsubstituted heteroaliphatic; substituted or unsubstituted aryl; substituted or unsubstituted heteroaryl; substituted or unsubstituted acyl; a resin; a suitable hydroxyl, amino, or thiol protecting group; or two R^{E} groups together form a substituted or unsubstituted 5- to 6-membered heterocyclic or heteroaromatic ring; and
R^{f} is, independently, hydrogen; cyclic or acyclic, branched or unbranched, substituted or unsubstituted aliphatic; cyclic or acyclic, branched or unbranched, substituted or unsubstituted heteroaliphatic; substituted or unsubstituted aryl; substituted or unsubstituted heteroaryl; substituted or unsubstituted acyl; a resin; a suitable amino protecting group; or R^{f} and R^{a} together form a substituted or unsubstituted 5- to 6-membered heterocyclic or heteroaromatic ring.

63-74. (canceled)


SEQUENCE LISTING (24 sequences)

SEQ ID NO: 1 - protein, 36 aa, Artificial Sequence
  Synthetic Polypeptide
ISKERERAILSTKPPGTFLLRFSESSKEGGVTFTWV

SEQ ID NO: 2 - protein, 34 aa, Artificial Sequence
  Synthetic Polypeptide
IKERRAILSTKPPGTFLLRFSESSKEGGVTFTWV

SEQ ID NO: 3 - protein, 34 aa, Artificial Sequence
  Synthetic Polypeptide
ISKRERILSTKPPGTFLLRFSESSKEGGVTFTWV

SEQ ID NO: 4 - protein, 34 aa, Artificial Sequence
  Synthetic Polypeptide
ISKERERILSKPPGTFLLRFSESSKEGGVTFTWV

SEQ ID NO: 5 - protein, 34 aa, Artificial Sequence
  Synthetic Polypeptide
ISKEREAILTKPPGTFLLRFSESSKEGGVTFTWV

SEQ ID NO: 6 - protein, 34 aa, Artificial Sequence
  Synthetic Polypeptide
ISKERRAILSTPPGTFLLRFSESSKEGGVTFTWV

SEQ ID NO: 7 - protein, 34 aa, Artificial Sequence
  Synthetic Polypeptide
ISKEERAILSKPPGTFLLRFSESSKEGGVTFTWV

SEQ ID NO: 8 - protein, 34 aa, Artificial Sequence
  Synthetic Polypeptide
ISKRERAILTKPPGTFLLRFSESSKEGGVTFTWV

SEQ ID NO: 9 - protein, 33 aa, Artificial Sequence
  Synthetic Polypeptide
ISKRERILSKPPGTFLLRFSESSKEGGVTFTWV

SEQ ID NO: 10 - protein, 33 aa, Artificial Sequence
  Synthetic Polypeptide
ISKRERILSTKPPTFLLRFSESSKEGGVTFTWV

SEQ ID NO: 11 - protein, 33 aa, Artificial Sequence
  Synthetic Polypeptide
ISKERERILSKPPTFLLRFSESSKEGGVTFTWV

SEQ ID NO: 12 - protein, 32 aa, Artificial Sequence
  Synthetic Polypeptide
ISKRERILSKPPTFLLRFSESSKEGGVTFTWV

SEQ ID NO: 13 - protein, 33 aa, Artificial Sequence
  Synthetic Polypeptide
ISKEREAILTKPPTFLLRFSESSKEGGVTFTWV

SEQ ID NO: 14 - protein, 33 aa, Artificial Sequence
  Synthetic Polypeptide
ISKEERAILSKPPTFLLRFSESSKEGGVTFTWV

SEQ ID NO: 15 - protein, 33 aa, Artificial Sequence
  Synthetic Polypeptide
ISKRERAILTKPPTFLLRFSESSKEGGVTFTWV

SEQ ID NO: 16 - protein, 14 aa, Artificial Sequence
  Synthetic Polypeptide
ISKERERAILSTKP

SEQ ID NO: 17 - protein, 12 aa, Artificial Sequence
  Synthetic Polypeptide
IKERRAILSTKP

SEQ ID NO: 18 - protein, 12 aa, Artificial Sequence
  Synthetic Polypeptide
ISKRERILSTKP

SEQ ID NO: 19 - protein, 12 aa, Artificial Sequence
  Synthetic Polypeptide
ISKERERILSKP

SEQ ID NO: 20 - protein, 12 aa, Artificial Sequence
  Synthetic Polypeptide
ISKEREAILTKP

SEQ ID NO: 21 - protein, 12 aa, Artificial Sequence
  Synthetic Polypeptide
ISKERRAILSTP

SEQ ID NO: 22 - protein, 12 aa, Artificial Sequence
  Synthetic Polypeptide
ISKEERAILSKP

SEQ ID NO: 23 - protein, 12 aa, Artificial Sequence
  Synthetic Polypeptide
ISKRERAILTKP

SEQ ID NO: 24 - protein, 22 aa, Artificial Sequence
  Synthetic Polypeptide
PGTFLLRFSESSKEGGVTFTWV